(19) United States
(12) Patent Application Publication
(10) Pub. No.: US 2010/0104505 A1
(43) Pub. Date: Apr. 29, 2010

(54) System and Method for Quantitative Molecular Breast Imaging

(76) Inventor: Michael K. O'Connor, Rochester, MN (US)

Correspondence Address: QUARLES & BRADY LLP, 411 E. WISCONSIN AVENUE, SUITE 2040, MILWAUKEE, WI, 53202-4497, US

(21) Appl. No.: 12/635,479

(22) Filed: Dec. 10, 2009

Related U.S. Application Data
(63) Continuation-in-part of application No. 12/515,369, filed on May 18, 2009.
(60) Provisional application No. 60/869,419, filed on Dec. 11, 2006.
(60) Provisional application No. 61/121,217, filed on Dec. 10, 2008.

(51) Int. Cl. A61K 51/00; A61B 6/00

(52) U.S. Cl. 424/1.11; 378/37; 600/431

(57) ABSTRACT

A system and method for performing quantitative lesion analysis in molecular breast imaging (MBI) using the opposing images of a slightly compressed breast that are obtained from the dual-head gamma camera. The method uses the shape of the pixel intensity profiles through each tumor to determine tumor diameter. Also, the method uses a thickness of the compressed breast and the attenuation of gamma rays in soft tissue to determine the depth of the tumor from the collimator face of the detector head. Further still, the method uses the measured tumor diameter and measurements of counts in the tumor and background breast region to determine relative radiotracer uptake or tumor-to-background ratio (T/B ratio).

### CROSS-REFERENCE TO RELATED APPLICATIONS

This application is a continuation-in-part of, claims the benefit of, and hereby incorporates by reference in its entirety U.S. patent application Ser. No. 12/515,369 filed on May 18, 2009, and entitled “System and Method for Quantitative Molecular Breast Imaging,” which claims the benefit of PCT Application No. US2007/086991 filed on Dec. 10, 2007, which claims the benefit of U.S. Provisional Patent Application Ser. No. 60/869,419 filed on Dec. 11, 2006. This application also claims the benefit of and hereby incorporates by reference in its entirety U.S. Provisional Patent Application Ser. No. 61/121,217 filed on Dc. 10, 2008, and entitled “Method and Apparatus for X-Ray Mammography/Tomosynthesis and Emission Mammography of the Breast.”

### STATEMENT REGARDING FEDERALLY SPONSORED RESEARCH

Not Applicable.

### BACKGROUND OF THE INVENTION

The invention relates to a system and method for obtaining quantitative information regarding breast images acquired using gamma cameras.

Screening mammography has been the gold standard for breast cancer detection for over 30 years, and is the only available screening method proven to reduce breast cancer mortality. However, the sensitivity of screening mammography varies considerably. The most important factor in the failure of mammography to detect breast cancer is radiographic breast density. In studies examining the sensitivity of mammography as a function of breast density, it has been determined that the sensitivity of mammography falls from 87-97 percent in women with fatty breasts to 48-63 percent in women with extremely dense breasts.

Diagnostic alternatives to mammography include ultrasound and magnetic resonance imaging (“MRI”). The effectiveness of whole-breast ultrasound as a screening technique does not appear to be significantly different from mammography. MRI has a high sensitivity for the detection for breast cancer and is not affected by breast density. However, since bilateral breast MRI is currently approximately 20 times more expensive than mammography, it is not in widespread use as a screening technique.

Another prior-art technology is positron emission mammography (“PEM”). This uses two, small, opposing positron emission tomography (“PET”) detectors to image the breast. The PEM technology offers excellent resolution; however, the currently available radiotracer (F-18 Fluoro deoxyglucose) requires that a patient fast overnight, the patient must have low blood levels (this is often a problem for diabetics), and after injection, the patient must wait 1-2 hours for optimum uptake of F-18FDG in the tumor. The high cost of these PET procedures coupled with the long patient preparation time reduces the usefulness of this procedure and makes it difficult to employ for routine breast evaluation.

Radionuclide imaging of the breast (scintimammography) with Tc-99m sestamibi was developed in the 1990s and has been the subject of considerable investigation over the last 10-15 years. This functional method is not dependent upon breast density. Large multi-center studies have shown the sensitivity and specificity of scintimammography in the detection of malignant breast tumors to be approximately 85 percent. However, these results only hold for large tumors and several studies have shown that the sensitivity falls significantly with tumor size. The reported sensitivity for lesions less than 10-15 mm in size was approximately 50 percent. This limitation is particularly important in light of the finding that up to a third of breast cancers detected by screening mammography are smaller than 10 mm. Prognosis depends on early detection of the primary tumor. Spread of a cancer beyond the primary site occurs in approximately 20-30 percent of tumors 15 mm or less in size. However, as tumor size grows beyond 15 mm, there is an increasing incidence of node positive disease, with approximately 40 percent of patients having positive nodes for breast tumors 2 cm in diameter. Hence, for a nuclear medicine technique to be of value in the primary diagnosis of breast cancer, it must be able to reliably detect tumors that are less than 15 mm in diameter. The failure of conventional scintimammography to meet this limit led to its abandonment as a useful technique in the United States.

In an attempt to overcome the limitation of conventional scintimammography, several small field-of-view gamma cameras have been developed that permit the breast to be imaged in a similar manner and orientation to conventional mammography. One commercial system for single photon imaging that is currently available is that manufactured by Dilon Technologies of Newport News, Va. Using a small detector and compression paddle, they reported a sensitivity of 67 percent for the detection of sub-10 mm lesions.

These systems employ a small gamma-ray camera that is attached to a mammography unit or to a stand-alone system in such a way that the gamma-ray camera is proximate to or in direct contact with a breast compression system. The system includes two identical opposing cadmium zinc telluride (“CZT”) detectors and performs planar imaging of the breast under compression. Recent clinical studies with the dual-head system have shown an increase in sensitivity to nearly 90 percent for lesions less than 10 mm.

Despite this improved percentage of success, the failure to identify lesions of any size can have significant consequences. Accordingly, it would be desirable to have a system and method to provide additional information to aid in the process of diagnosis, analysis, and treatment planning.

### SUMMARY OF THE INVENTION

The present invention overcomes the aforementioned drawbacks by providing systems and methods for performing quantitative tumor analysis using information acquired with a dual-headed molecular breast imaging system. Specifically, the present invention provides systems and methods to utilize the information available in planar dedicated breast imaging to provide previously unavailable information sets to aid in the diagnosis and biopsy of the site. In particular, the present invention provides a method for accurately determining the size, depth to the collimator, and relative tracer uptake of a tumor.

In order to measure the diameter of a tumor, the present invention uses the shape of the pixel intensity profiles through each tumor to determine tumor diameter. Also, the method uses knowledge of compressed breast thickness and the attenuation of gamma rays in soft tissue to determine the depth of the lesion from the collimator face of the detector. Further still, the present invention uses the measured lesion diameter and measurements of counts in the lesion and background breast region to determine relative radiotracer uptake or tumor-to-background ratio (T/B ratio).

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is an illustration of a molecular breast imaging system for use with the present invention;

FIG. 2 is a flowchart setting forth the steps of a method for determining a tumor size using the system of FIG. 1 and in accordance with some embodiments of the present invention;

FIG. 3 is a schematic representation of a user interface for determining tumor size in accordance with some embodiments of the present invention;

FIG. 4 is a flowchart setting for the steps of a method for determining tumor depth and relative radiotracer uptake in accordance with some embodiments of the present invention;

FIG. 5 is a graph showing ROI diameters that produced a minimum error in measured depth for tumor diameters of 4-20 mm and a range of breast thicknesses;

FIGS. 6A-6D show the progression of molecular breast imaging (“MBI”) systems, ranging from the first detector mounted on a modified thyroid uptake probe stand, to today's dual-head detector system incorporated into a modified mammographic gantry;

FIG. 7 is a schematic representation of an experimental arrangement used to simulate clinical MBI studies;

FIG. 8 is a graph showing the energy spectra from patient studies, experimental phantom study, and Monte Carlo simulation;

FIG. 9 is a graph showing the energy spectrum from MC simulation showing the contribution of the various components of the spectrum, with the vertical lines showing limits of the standard energy window;

FIG. 10 is multiple images of a breast phantom containing tumors about 4-9 mm in diameter and imaged at depth of about 1-5 cm with a tumor/background ratio of about 10:1;

FIG. 11 are images of MBI studies in patients with about 3-17 mm breast tumors;

FIG. 12 is a graph showing simulated energy spectra from a distributed Tc-99m source and a gamma camera;

FIG. 13 is a user interface layout for a program to perform image quality control prior to generation of a geometric mean image;

FIG. 14 is a graph showing the correlation between true and measured tumor diameter using various percentages of the width of the tumor profile ; and

FIG. 15 is a schematic representation of conventional parallel-hole collimation compared to slant-hole collimation.

### DETAILED DESCRIPTION OF THE INVENTION

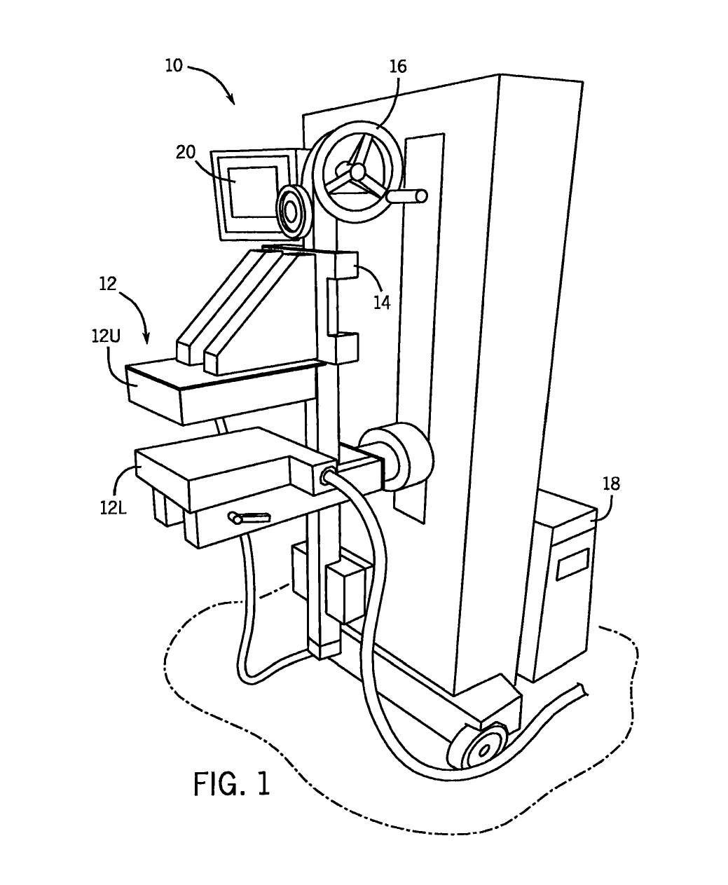

Referring to the Figs., and in particular FIG. 1, a molecular breast imaging (“MBI”) system 10 includes two opposing cadmium zinc telluride (“CZT”) detectors (detector heads) 12. In particular, the detector heads 12 include an upper detector head 12U and a lower detector head 12L. Each detector head 12U, 12L is, for example, 20 cm by 16 cm in size and mounted on a modified upright type mammographic gantry 14. In accordance with one embodiment, the detector heads 12 are LumaGEM 3200S high-performance, solid-state cameras from Gamma Medica having a pixel size of 1.6 mm. LumaGEM is a trademark of Gamma Medica, Inc. Corporation of California.

The relative position of the detector heads 12 can be adjusted using a user control 16. Specifically, the detector head assemblies 12 are, preferably, designed to serve as a compression mechanism. Accordingly, this system configuration reduces the maximum distance between any lesion in the breast and either detector head 12 to one-half of the total breast thickness, potentially increasing detection of small lesions without additional imaging time or dose. The MBI system 10 includes a processor 18 for processing the signals acquired by the detector heads 12 to produce an image, which may be displayed on an associated display 20.

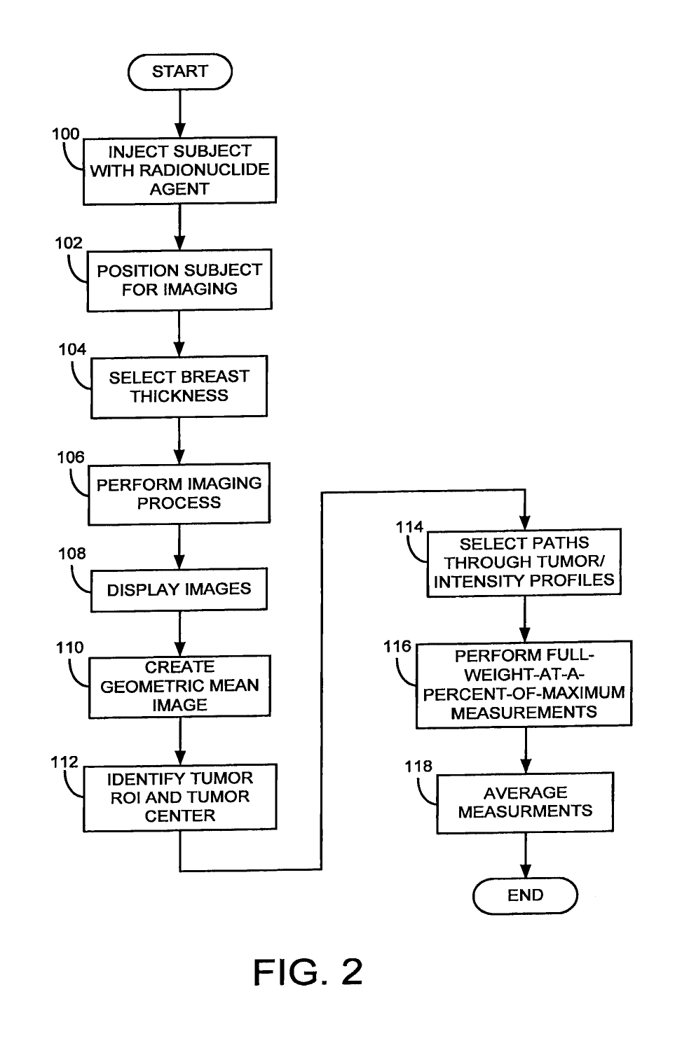

Referring to FIGS. 1 and 2, a process in accordance with the present invention begins at process block 100 by injecting a subject with a radionuclide imaging agent such as Tc-99m sestamibi (20 mCi/injection). The subject is then positioned for imaging at process block 102. Specifically, the subject is positioned so that a breast is arranged between the detector heads 12. The detector heads 12 are then adjusted using the user control 16 to lightly compress the breast between the upper detector head 12U and lower detector head 12L to improve image contrast and reduce motion artifacts. The compression amount is approximately ⅓ that of conventional mammography and is typically improves contrast and reduces motion artifacts.

Once the subject is properly positioned, the breast thickness is selected at process block 104. Specifically, the breast thickness may be automatically determined based on the relative position of the upper detector head 12U and the lower detector head 12L or an operator may enter the breast thickness through a user interface, the display 20.

At approximately 5 minutes post-injection, the breast is imaged at process block 106. An image is acquired by each detector head 12U, 12L of each breast at multiple views. For example, an image may be acquired in craniocaudal (CC) and mediolateral oblique (MLO) positions for 10 minutes per view. Furthermore, it is contemplated that imaging may be performed at multiple directions using both the craniocaudal and mediolateral oblique breast views to obtain a three-dimensional estimate of tumor size.

At each view, the images are simultaneously acquired by the upper detector head 12U and the lower detector head 12L. Thus, for each breast, multiple sets of data are acquired that are processed by the processor 18 and then shown to the operator on the display 20 or other viewing locality at process block 108. At a minimum, it is contemplated that the operator visually evaluates the four images (lower CC, upper CC, lower MLO, upper MLO) acquired of each breast.

In addition to the images described above, it is contemplated that at least one additional image may be generated at process block 110 that is a geometric mean image of the two opposing images. As a lesion moves deeper in the breast or farther away from a given detector head 12U or 12L, the diameter of the lesion increases due to the isotropic nature of the emitted photons. For example, a lesion closer to the lower detector head 12L appears smaller in the image acquired by the lower detector head 12L than in the image acquired by the upper detector head 12U. The geometric mean image of the two opposing images created at process block 110 provides a consistent lesion size on which to perform a measurement of the size of an identified tumor for a given breast thickness. Therefore, within the geometric mean image, a given tumor has a contrast indicative of the tumor being positioned in the middle of the breast, at half the total compressed breast thickness.

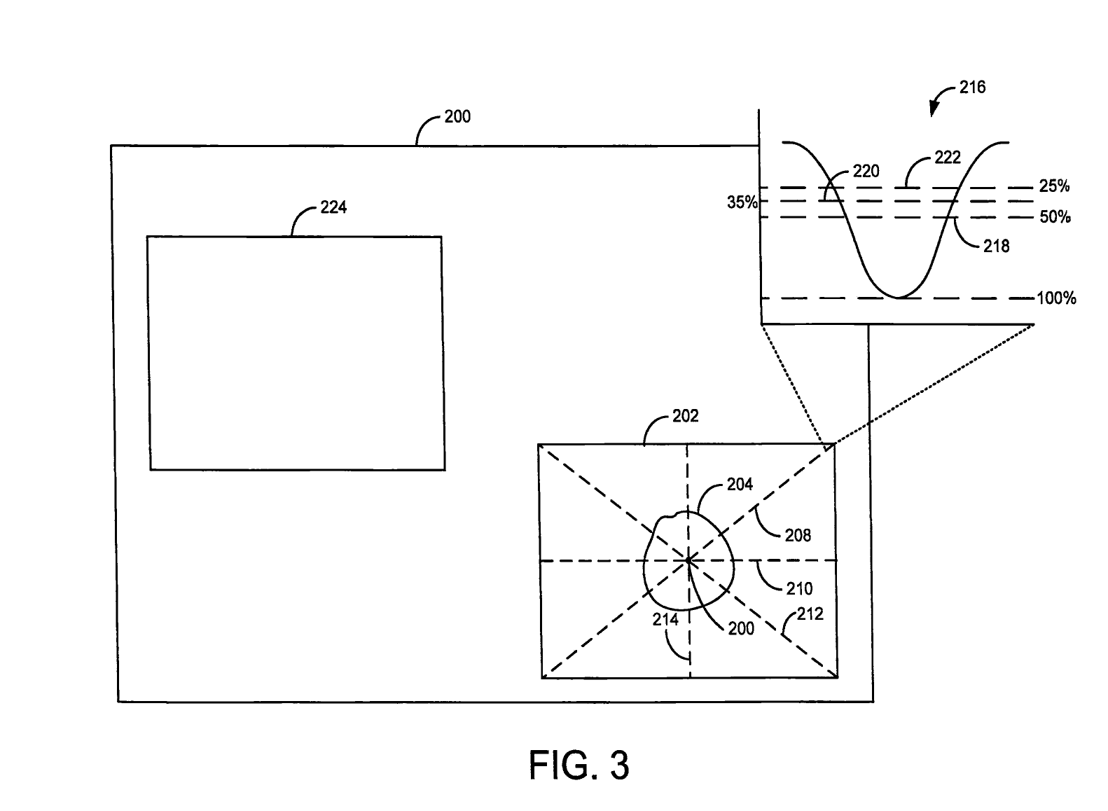

Using these images, any tumors appearing in the images are identified at process block 112 by selecting a tumor region of interest (ROI) including the tumor and indicating the center 206 of the tumor 204. For example, referring to FIG. 3, an image 200 may be displayed for an operator to select a tumor ROI 202 including evidence of a tumor 204 in the displayed image 200. Also, it is contemplated that the system may attempt to automatically identify the tumor(s) 204 within a given image or images 200 and select a preliminary ROI 202.

Referring now to FIGS. 2 and 3, with this information entered, a plurality of paths 208-214 that extend through the tumor locations/centers 204/206 are selected at process block 114. In accordance with one embodiment, at least four paths at 0, 45, 90, and −45 degrees are obtained. However, the accuracy of the size measurement can be improved by using a larger number of paths through the tumor 202 and corresponding intensity profiles. That is, as illustrated in FIG. 3, these paths 208-214 have corresponding intensity profiles 216. For each intensity profile 216, a number of full-width-at-a-percentage-of-maximum measurements 218-22 are performed at process block 116. In particular, full widths of each profile at a variety of percentages of the maximum value are measured at process block 116. For example, the full width of each profile at 10, 15, 20, 25, 30, 35, 40, and 50 percent of the maximum value can measured. However, such a large sample is not typically necessary and the full widths at, for example, 25, 35, and 50 percent may be used. Regardless of the specific number of measurements obtained, the measurements are averaged at process block 118 to provide an average measurement metric indicating the diameter/size of the identified tumor 204.

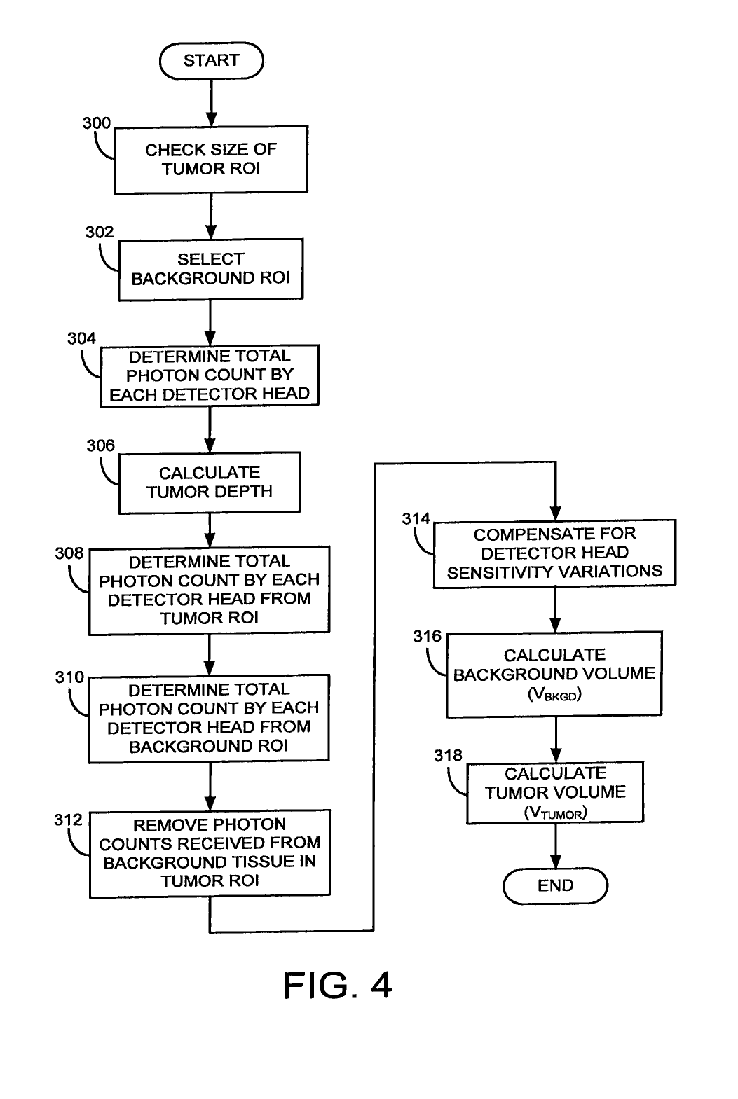

Continuing with respect to FIGS. 1 and 4, the present method described with respect to FIG. 2 can be expanded to determine the depth of an identified tumor with respect to the lower (or upper) collimator face. The method begins at process block 300 by checking the size of the tumor ROI 202 selected at process block 112 of FIG. 2. Specifically, the ROI size applied to each tumor must be large enough to include nearly all of the photon counts received from the tumor 204 by both the upper and lower detector heads and to yield the corresponding images.

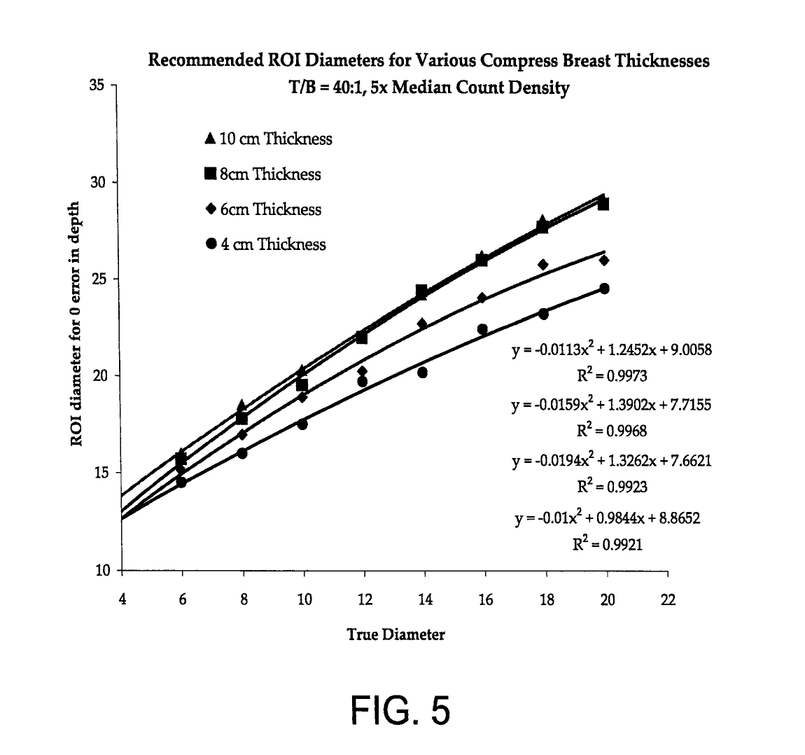

To test the appropriate ROI size, the error in measured tumor depth was plotted as a function of ROI diameter. For each tumor diameter, there is a range of appropriate ROI diameters that produce a low (±1 mm) error in tumor depth. The zero crossing of each curve was used to determine the best ROI diameter to use for tumor depth measurement. FIG. 5 shows the ROI diameters that produced the minimum error in measured depth for tumor diameters of 4-20 mm and breast thicknesses of 4, 6, 8, and 10 cm.

To facilitate more precise placement of tumor ROls, images can be interpolated by factors of 10 using a linear interpolation algorithm to resample the images with an adjusted pixel size, for example, 0.16×0.16 mm². The linear algorithm calculates the resampled pixel intensities by examining the neighboring intensities of the original image and integrating them based on their proportional distance from the projected resampling position.

Referring again to FIGS. 3 and 4, once an appropriate size of the tumor ROI 202 has been confirmed, a background ROI 224 is selected at process block 302. Specifically, the reference or background ROI 224 is selected to have the same size dimensions of the tumor ROI 202, but include only background tissue that is substantially free of tumor(s).

Once the tumor and background ROls 202, 224 have been selected, at process block 304, the number of photons received at each detector head during the imaging process is determined. The photon counts made by the lower detector head 12L and upper detector head 12U are represented as N_{L} and N_{U}, respectively, as follows:

N_{L}=Nₒ·exp(−μd) Eqn. (1);

N_{U}=N_{O}·exp(μ(t−d)) Eqn. (2);

where N_{O} is the number of unattenuated photons determined at process block 304, p is a known attenuation coefficient of soft tissue (0.153 cm⁻¹), t is compressed breast thickness determined at process block 104 of FIG. 2, and d is tumor depth to be determined. Using these photon counts, a tumor depth calculation is performed at process block 306 by solving for d in Eqns. (1) and (2). Specifically, Eqns. (1) and (2) are solved for N_{O} and then set equal to each other to yield the following equation for tumor depth, d:

$$d = \frac{μ t - ln ( \frac{{N}_{L}}{{N}_{U}} )}{2 μ} . \qquad Eqn . ( 3 )$$

Thereafter, a further refined depth measurement can be provided by removing photon counts provided by background structures in the ROI. Specifically, the sum of photon counts received from the tumor ROI identified at process block 212 of FIG. 2 and confirmed at process block 300 of FIG. 4 is calculated for each detector head at process block 308. Thereafter, at process block 310, the sum of photon counts received from the background ROI is calculated for each detector head at process block 310. With this additional information, Eqn. (3) can be modified to account for photon counts only coming from the tumor. Specifically, at process block 312, the total background photon counts received from the background ROI is subtracted from the total photon counts received from the tumor ROI to remove the photon counts from the tumor ROI that are attributable to background tissue. Also, at process block 314, a correction can be applied to the photon counts from upper detector head 12U (or, alternatively, to the lower detector head 12L if the upper detector head 12U is used as the reference frame from which the depth measurement is made) to adjust for possible differences in detector sensitivity. The steps taken at process bocks 312 and 314 are achieved by modifying Eqn. (3) to yield the following:

$$d = \frac{μ t - ln ( \frac{{T}_{L} - {B}_{L}}{( {T}_{U} - {B}_{U} ) \cdot \frac{{B}_{L}}{{B}_{U}}} )}{2 μ} ; \qquad Eqn . ( 4 )$$

where T_{L} and T_{U} are the sum of photon counts received from an identical ROI placed on the tumor in the images provided by the lower detector head 12L and upper detector head 12U, respectively, and B_{L} and B_{U} are the sum of photon counts received from an ROI of equal size placed in a uniform background breast tissue region of the image provided by the lower detector head 12L and upper detector head 12U, respectively.

Additionally, using the ROI size determined as described above, photon counts in the tumor and background ROls can be used to calculate a tumor to background (T/B) uptake ratio. To do so, the process continues by calculating a background volume (V_{bkgd}) at process block 316. Specifically the area of the background ROI selected at process block 302 is multiplied by the thickness of the breast determined at process block 104 of FIG. 2 to yield the background volume. Then, a tumor volume (Vₜᵤₘₒᵣ) is calculated at process block 318 using the tumor size/diameter calculated as described above with respect to FIG. 2. The T/B ratio is therefore calculated as follows:

$$T / B Ratio = \frac{\sqrt{( {T}_{L} \cdot {T}_{U}} - \sqrt{( {B}_{L} \cdot {B}_{U}}}{\sqrt{( {B}_{L} \cdot {B}_{U}}} \cdot \frac{{V}_{Bkgd}}{{( {V}_{Tumor} )}^{F}} ; \qquad Eqn . ( 5 )$$

where F is a constant of 0.99 that was empirically determined to provide a more accurate measure of T/B ratio. In accordance with one embodiment of the invention, it is contemplated that the tumor volume may be estimated by assuming a spherical tumor shape using the tumor diameter determined as described above with respect to FIG. 2. However, as described above, to more accurately determine the volume of non-spherical lesions, a higher number of intensity profiles extending in multiple directions through the tumor, using both the craniocaudal and mediolateral oblique breast views, could be used to obtain a better estimate of tumor size. In any case, Eqn. (5) is the ratio of the geometric mean of the tumor regions to the geometric mean of the background regions with corrections for differences in the ROI volumes.

It is noted that one advantage of the present invention is that the specific pixel size used results in statistically insignificant changes in the measured diameter, depth, and T/B ratio. However, both the depth and the T/B ratio measurements are dependent on an accurate measurement of tumor size/diameter. While Eqn. (4) does not directly depend on the size/diameter measurement, the ROI size used to perform the depth measurement is determined from the previously measured tumor size. Also, as described above with respect to Eqn. (5), the T/B ratio is directly dependent on tumor volume, which is calculated using the measured tumor size/diameter.

To quantify the dependence of depth and T/B ratio calculations on the size/diameter measurement, calculations for depth and T/B ratio were performed on a set of Monte-Carlo simulated, dual-head images after setting the tumor diameter to 1 mm greater and 1 mm less than the known true diameter for each tumor. These images were acquired with a 6 cm breast thickness, a tumor depth of 2 cm from the lower detector, and a T/B of 40:1. The expected change in T/B measurement was calculated by manipulating Eqn. 5 as follows:

$$T / B Ratio ∝ \frac{1}{{( {V}_{Tumor} )}^{F}} = \frac{1}{{[ \frac{4}{3} {π ( \frac{d}{2} )}^{3} ]}^{.99}} ; \qquad Eqn . ( 6 )$$

which shows that T/B ratio is inversely proportional to the tumor volume raised to the factor F=0.99. Therefore, T/B ratio is inversely proportional to diameter, d, cubed and raised to the power of 0.99 as shown in Eqn. (6). The change in T/B ratio for a given fraction of the true diameter can thus be calculated as follows:

$$Change in T / B Ratio = \frac{1}{{( {k}^{3} )}^{.99}} ; \qquad Eqn . ( 7 )$$

where k is the fraction of the true diameter. For example, if tumor diameter is underestimated by 5 percent, k=0.95 and the change in T/B is 1.165, or T/B is overestimated by 16.5 percent. Nevertheless, the percent error in the calculated T/B ratio was tested to show the absolute average error in T/B ratio can be controlled to be less than 5 percent for all breast thicknesses, except at the T/B ratio of 10:1, where error was nearly 9 percent at a 4 cm breast thickness.

Therefore, while depth measurements are nearly unchanged for small errors in the diameter measurement, T/B ratio can be significantly affected by a large percent error in the diameter measurement. However, as noted above, the accuracy of the diameter measurement can be improved by using more than a larger number of intensity profiles through the tumor.

Additional description and significance of the invention will be expanded on below.

The current modalities for imaging the breast suffer from a number of limitations that either reduce the sensitivity of the modality, or are expensive to perform, limiting their clinical value. This application focuses on a new technique for breast imaging with both short and long term goals. The short term goal is the development of an alternative screening technique to mammography, particularly for women with dense breasts. For this to be achieved, the short term goal may include demonstrating a high sensitivity for the detection of small (e.g., less than about 1 cm) breast lesions. The long term goal is the development of the instrumentation and software which is useful for the growth of molecular imaging of the breast. There are a large number of radiopharmaceuticals that have potential applications in breast imaging, however, in the absence of the appropriate technology, the true potential of these agents cannot be realized. Hence, the long term goal is the development of the appropriate technology that will give investigators the tools for molecular imaging of the breast.

Background and Significance

Breast cancer is a major health problem for women worldwide and is the second most common cancer in the U.S. The incidence of breast cancer is increasing at approximately 3% per annum and about 1 in 9 women will develop invasive breast cancer during her lifetime (1). Although breast cancer incidence rates are rising, mortality rates are falling, indicating both a) an increased awareness resulting in earlier and increased detection through screening, and b) improved treatment outcomes. Early detection of a primary cancer is of paramount importance as treatment of the tumor when it is small significantly reduces morbidity and mortality.

Screening Mammography

Screening techniques (primarily mammography) have been shown to result in earlier diagnosis and up to about 25% to about 30% reduction in the relative risk of dying from breast cancer in women over the age of about 50 (2). Despite the demonstrated benefit of mammography, this technique has limitations in clinical practice (3-5). While it has a high sensitivity in women with fatty breasts, it is less reliable in patients with radiographically dense breasts, breast implants, or following breast surgery (6). Numerous studies have demonstrated that the sensitivity of mammography is reduced in radiographically dense breasts (10-14). In one large prospective study of screening mammography, the sensitivity in patients classified as having extremely dense breast tissue was only about 44% (14).

This inverse relationship between breast density and the sensitivity of mammography has several implications. Firstly, while breast density decreases after menopause, the rate of fatty involution after menopause appears to be decreasing (15). This trend has been seen even in patients not receiving hormone therapy, and may be related to changes in childbearing patterns. A recent study found that about 25% of women aged about 50-69 had a dense mammographic breast pattern (16). Thus, an increasing proportion of women may be at risk for missed cancers on screening mammography as a result of breast density. Secondly, in addition to reducing the sensitivity of mammography, breast density is an independent risk factor for the development of breast cancer. Wolfe first described an association between a qualitative classification of dense mammographic patterns and an increased risk of breast cancer. Eleven other studies have confirmed this association: most of these studies found that the relative risk of breast cancer was at least quadrupled for women with the most breast density when compared with women with the least. The decreased sensitivity and specificity of mammography in women with dense breasts, and the increased risk conferred by breast density underscore the importance of an alternative form of imaging in women with dense breast parenchyma, particularly those who have risk factors in addition to breast density.

In the above-referenced study of factors contributing to mammography failure in women about 40-49 years of age, breast density was a factor, but rapid tumor growth explained about 31% of the decreased sensitivity of mammography. Interval cancers represent a heterogeneous group comprising: 1) cancers missed on prior mammogram but present in retrospect; 2) cancers present but mammographically indistinct; and 3) cancers that were clearly not visible on the prior mammogram and so appear to have arisen in the interval between screening mammograms. In regard to the category of cancers that clearly were not visible on the prior mammogram, several studies have shown that these tumors are more likely to have high proliferation (either by mitotic count or Ki-67 antigen expression), high histological grade, and high nuclear grade than tumors detected on screening mammography. An imaging modality that capitalizes on this high molecular activity rather than relying on anatomic distinctions between tumor and normal breast tissue might improve detection of true interval cancers.

Digital Mammography

Digital mammography offers the potential to be significantly better than conventional mammography in the early detection of breast cancer [26-28]. Clinical trials of full-field digital mammography to date have compared sensitivity, specificity, and receiver operating characteristic (ROC) curves of digital to screen-film mammography, typically in paired studies of the two modalities. The largest study to date has been the National Cancer Institute sponsored American College of Radiology Imaging Network (ACRIN) DMIST [NEJM article] study. This study reported the sensitivity and specificity of both conventional and digital mammography in over about 49,000 women. Overall the sensitivity of digital and film mammography was about 70% and about 66% respectively at about 1 year follow-up. These sensitivities dropped to about 41% at follow-up of about 1 year+100 days. In women with heterogeneously dense or extremely dense breasts, results showed that at one year follow-up digital mammography had a significantly higher sensitivity of about 70% compared to a sensitivity of about 55% for conventional film mammography. However at a follow-up of about 1 year+100 days, the sensitivity of both techniques had dropped to approximately 37%. Hence while digital mammography does improve the sensitivity of mammography in women younger women and those with dense breasts, the use of a purely anatomical modality alone may never be able to achieve the desired sensitivity.

Ultrasound and MRI

Both ultrasound and MRI have been studied in the dense breast patient population. Several single-center studies of whole-breast bilateral sonography have been shown to depict nonpalpable invasive breast cancers not visible on mammography, particularly in dense breasts. In the largest series of screening bilateral whole breast sonography to date, Kolb et. al. analyzed 27,825 screening sessions that included mammogram and physical exam as well as ultrasound. Overall, the study showed that mammography and ultrasound had similar sensitivities (e.g., about 77.6% and about 75.3% respectively) and specificities. In women with dense breasts ultrasound appeared to have a higher sensitivity, especially in women with extremely dense breasts where the sensitivity of ultrasound was about 76% compared to about 48% for mammography. The advantages of ultrasound include the absence of ionizing radiation, the absence of painful compression, and the lower cost relative to breast MRI. However, ultrasound is highly dependent on operator experience, and the studies done thus far reflect significant operator expertise that may not be reproducible in other settings. Furthermore, the quality of the ultrasound varies with the type of equipment used. There are no standardized techniques with respect to transducer frequency, positioning of the patient, scan planes, or setting of focal zones. Validated criteria do not yet exist for following incidental masses seen only on sonography, contributing to the high biopsy rate. These issues are currently being addressed in the first multicenter protocol to assess the efficacy of screening breast sonography (American College of Radiology Imaging Network 6666 Trial).

MRI has not been studied in the general population as a screening tool, but has been studied in high risk populations. Five prospective studies of screening MRI have been done in women at increased risk of breast cancer on the basis of a known or suspected mutation in a breast cancer susceptibility gene or a calculated lifetime risk of breast cancer exceeding about 15%. Given the small number of cases with breast cancer in these studies (ranging from about 3 to about 45 cases), estimates of sensitivity are not precise. Nevertheless, all five studies did report higher sensitivity for breast MRI than for mammography. In the largest of these studies, 1909 women in the Netherlands with a cumulative lifetime risk of breast cancer of about 15% or more were screened every year by mammography and MRI. The sensitivity of mammography was about 40%, compared to a sensitivity of about 71% for MRI. However, the specificity of MRI in this study was lower than for mammography (about 89.8% vs. about 95%), and screening with MRI led to twice as many unneeded additional examinations and about 3 times as many unneeded biopsies as compared to screening mammography. This may be explained by the fact that the both malignant and benign lesions have similar high water and cellular content with a high degree of fibrosis. Enthusiasm for MRI as an accurate breast cancer screening technique remains somewhat elevated in spite of the poor specificity of MRI [40]. As observed in other applications of MRI, critics have questioned the cost-effectiveness of breast MRI and whether or not this expensive technique can provide information that will ultimately impact patient outcomes. In addition to long examination times, inability to visualize microcalcifications, and poor specificity, the major disadvantage of MRI is the high cost. While MRI has gained some acceptance as a screening modality in women at the highest level of risk (women with the BRCA gene mutation), the high cost will likely limit its application to women with a lesser degree of increased risk.

Molecular Imaging—Positron Emission Tomography (PET)/Mammography (PEM)

One of the most significant components of molecular imaging is nuclear imaging. This modality not only uses radioisotopes for diagnosing of diseases but also for research. Nuclear imaging can further our understanding of a diseases process or behavior of a drug in-vivo. Breast imaging utilizes molecular imaging in many aspects and its scope is outlined below.

Several radiopharmaceuticals have demonstrated a complementary role to x-ray mammography (XMM), based on the fact that malignant cells have an increased metabolic activity in order to drive their high proliferation rate [45, 46]. By far the leading metabolic agent is 18F-fluoro-2-deoxy-D-glucose (FDG). The higher metabolic activity of the tumor enhances local uptake of F-18 FDG enabling detection by the PET scanner. A number of studies involving FDG-PET have been conducted and reported in the literature. Sensitivity and specificity for the staging of breast cancer using FDG-PET imaging have been estimated at about 92% and about 94%, respectively [7, 47]. However while PET works well for disease staging, the limited spatial resolution of wholebody PET scanners, coupled with attenuation through the thorax and the off-axis position of the breast limits its usefulness for the detection of primary breast disease, particularly lesions less than about 10 mm in size. These limitations have resulted in research and development projects in the field of dedicated breast PET scanners, now known as the field of Positron Emission Mammography, or PEM.

There have been many different PEM devices developed in the recent years all with promising results. Unfortunately not many have progressed to clinical trials and even fewer devices have been FDA approved. There are currently two types of dedicated PET prototypes being developed, a well-type ring scanner into which the pendulous breast is suspended, and an opposing pair of planar detectors operating in coincidence [51, 52]. Both systems claim less expensive installation and function, compact size, easier operation, less radiotracer dose, and better spatial resolution compared with full-ring, whole body PET scanners. Naviscan PET Systems (Rockville, Md.) is one of the few companies that has an FDA approved detector and has conducted a limited pilot study. A small study of 44 patients was performed [53] to assess the accuracy of PEM in newly diagnosed breast cancer patients. The majority of the lesions were identified on PEM (about 89%, 39/44) and 4/5 incidental breast cancers were found, 3 of which were not seen by any other imaging modalities. Of 19 patients undergoing breast-conserving surgery, PEM correctly predicted about 75% (6/8) patients with positive margins and 100% (11/11) with negative margins. While these results are promising and warrant a larger clinical trial, the advantages of PEM for screening are diminished by the cost of the procedure (comparable in cost to PET), the patient preparation (patients should fast for about 4 hours and sit in a quite room for about an 1 hour post injection), and the goal of controlling blood glucose levels which can be problematic in patients with diabetes. Nevertheless, the ease with which new radiotracers can be developed insures that this technology will find a useful place in breast imaging.

Molecular Imaging—Scintimammography

Functional imaging is not new to breast tumor localization. For several years researchers have attempted to advance breast scintigraphy (also known as “scintimammography” or SMM) using a number of different radiopharmaceuticals and conventional scintillation gamma camera technology. SMM with technetium Tc99m sestamibi has been shown to be a good complementary technique to mammography. The value of Sestamibi for breast tumor imaging was incidentally discovered during myocardial perfusion studies. Physicians noted areas of Sestamibi activity within breast tissue during a cardiac study which later proved to be breast cancer. The mechanism of Tc-99m Sestamibi uptake in cancerous cells is still not well understood, however experimental studies have shown that intercellular concentration of this agent in carcinoma cells lines was nearly 9 times higher than in normal, non-myocardial cells [55]. Experimental data indicate that intracellular uptake seems to primarily depend on tissue perfusion and mitochondrial activity [56].

During the past fifteen years, numerous studies have evaluated the performance of Tc99m sestamibi SMM for the diagnosis of breast cancer. In a review of over about 20 studies published between 1994 and 2000, Khalkhali and Vargas (7) reported an average sensitivity and specificity of about 75.4% and about 82.7% respectively. Tc-99m sestamibi is an approved agent for breast imaging and current indications are for the evaluation of breast cancer in patients in whom mammography is non-diagnostic, equivocal, or difficult to interpret, to assist in identifying multicentric and multifocal carcinomas, and in the evaluation of the effectiveness of neoadjuvant chemotherapy for breast carcinoma.

Despite the availability of reimbursement for SMM, this technique has never become a routine clinical tool. While the high sensitivity and specificity would indicate that SMM is a useful tool for the detection of breast cancer, a more detailed look at the data reveals a significant problem in the detection of small breast lesions. A large U.S. multicenter trial in 650 women reported sensitivities of about 48% and about 74% for lesions less than about 1 cm and equal to about 1 cm or greater respectively (28). A smaller European multi-center trial in 246 women reported similar sensitivities of about 40% and about 82% for lesions less than about 1 cm and equal to about 1 cm or greater respectively (26). Two large single-center studies by Tofani et al (15) and Spanu et al (29) reported sensitivities of about 48% and about 45% respectively for lesions less than about 1 cm. This failure to reliably detect small breast tumors has proved to be a major obstacle to its clinical acceptance, as early detection is one of the factors known to reduce the mortality rate. Consequently, SMM has failed to develop as a useful imaging technique in the detection of breast cancer. It is rarely used in clinical practice in the U.S. and the manufacturer of sestamibi ceased marketing of the radiopharmaceutical for breast imaging in 2005.

Detection of lesions less than about 1 cm in size is a major challenge for any technique employing general purpose gamma cameras. In order to attain improved performance for smaller lesions, dedicated small-field-of-view, high-performance gamma cameras are useful. These cameras can be designed to allow energy discrimination between the faint primary emissions of a small tumor and the stronger scattered emissions from the heart and thorax. Semi-conductor based gamma cameras using Cadmium Zinc Telluride (CZT) meet this design criterion. In the following section, preliminary results will be presented from an ongoing clinical trial at the Mayo clinic that demonstrate the impressive scatter rejection and lesion detection capabilities of this material.

Molecular Breast Imaging

It is useful to look at the reasons why scintimammography fails to detect small lesions. Conventional gamma cameras have a large dead space of between about 5-10 cm between the edge of the detector and the edge of the collimator field of view. This large dead space prevents us from imaging the patient with the gamma camera as we would in mammography. Consequently, conventional scintimammography is performed with the patient prone and the detector positioned laterally in close proximity to the pendulant breast. Scopinaro et. al. (18) reported the average thickness of the pendulant breast to be about 16 cm. In contrast, if the breast is imaged in a standard cranio-caudal view with light compression, they found that breast thickness was reduced to approximately 4 cm. This 4 fold reduction in breast thickness has a dramatic effect on the visibility of small breast lesions. However, in order to be able to image the breast in the cranio-caudal position with the gamma camera, the dead space between the edge of the detector and edge of the collimator should be minimized. This is not technically possible with conventional single crystal sodium iodide-based gamma cameras.

Several laboratories have been working toward the development of detectors optimized for breast imaging that permit the gamma camera to be positioned close to the breast as in mammography. Most of these detectors have utilized multi-crystal arrays of Cesium Iodide or Sodium Iodide crystals coupled to position sensitive photomultiplier tubes or photodiodes. Although many of these systems have poorer energy resolution than conventional gamma cameras, preliminary clinical results from some of these systems have shown a significant improvement in the detection of malignant breast lesions smaller than about 1 cm. Scopinaro et. al. developed a prototype breast camera with about a 12.5 cm field of view. Although the small field of view of this camera made it impractical for routine clinical use, they found an increased sensitivity from about 50% to about 81% for detecting breast cancers smaller than about 1 cm. A study by Brem et. al. using a multicrystal sodium iodide-based gamma camera adapted for breast imaging found that the sensitivity for detecting breast cancers less than about 1 cm was about 47% for conventional scintimammography and about 67% for the dedicated breast camera.

More recent studies have reported on the use of semiconductor-based gamma cameras, using Cadmium-Zinc-Telluride (CZT). These cameras have excellent energy resolution of about 4%-7%, compared to about 10% for conventional gamma cameras and up to about 20% for multicrystal cameras (ref). This improvement in energy resolution can reduce image scatter and contrast, permitting visualization of smaller lesions. In addition, current CZT detectors have a discrete intrinsic spatial resolution as small as about 1.6 mm. This is significantly better than the typical intrinsic resolution of about 3.5 mm in conventional systems. The presence of minimal dead space (e.g., about 8 mm) at the edge of a CZT detector coupled with the improvements in energy and spatial resolution make this technology the ideal tool for the development of a dedicated breast imaging system.

In a study by Coover et. al, 37 patients who had dense breasts, a family or personal history of breast cancer and no suggestive clinical or mammographic findings underwent scintimammography using both a standard gamma camera and a CZT-based gamma camera dedicated for breast imaging. Dedicated breast camera results were positive in about 13.5% (5/37) of patients. Biopsy of these 5 patients yielded 3 carcinomas, only one of which was detectable using a standard gamma camera.

At Mayo Clinic, work has been started on the development of a dedicated breast imaging system using a prototype dual-headed CZT camera system in 2001. Since this technology does not scintillate, the term SMM was inappropriate and we have used the term Molecular Breast Imaging (MBI) in association with the use of CZT detectors in breast imaging. The initial studies in patients with suspected breast cancer, demonstrated a sensitivity of about 77% for tumors less than about 1 cm in size and represented about 30% absolute improvement in sensitivity over values previously reported with conventional gamma cameras. Since then we have continued to refine the technique of MBI with CZT detectors. Work in the laboratory on energy resolution, collimation and gantry design has led to more optimal detector configurations for breast imaging. In September 2005 we constructed the first dual-head CZT detector in the world. This system uses 2 small field of view detectors to provide opposing views of the breast and increases the ability to detect small structures by reducing the potential distance of any lesion from the detector face. Preliminary results from this system in 70 patients have shown a sensitivity of about 90% for tumors less than about 1 cm in size. In addition, Monte Carlo simulations have demonstrated the potential of a dual-head design for absolute quantification of tumor uptake in the breast. This parameter may aid in distinguishing between benign and malignant processes as well as be a valuable tool for researchers who wish to quantitate the relative uptake of different radiopharmaceuticals in the breast.

From the above review, we believe that the current data indicate that mammography is a sub-optimal screening technique in women with dense breasts. Ultrasound has failed to show promise as an alternative technique and MRI, while demonstrating a high sensitivity, is not a cost-effective solution in this patient population. The results obtained with small semiconductor based gamma cameras over the last 4 years indicate that MBI may be as sensitive as MRI of the breast while being considerably more cost-effective. To the best of our knowledge, Mayo Clinic is the only institution in the world currently operating a dual-head CZT detector system and this has placed us in a unique position to appreciate the potential for this technology and to understand the future design improvements for a MBI system. In co-operation with Gamma Medica-Ideas, we believe that we possess the clinical experience and detector technology to create a true clinically functional dedicated breast imaging system.

Future Molecular Imaging Agents

The results from studies have demonstrated that sestamibi is a significantly better radiopharmaceutical for tumor imaging in the breast than previously believed. It is believed that the failure to recognize the potential value of sestamibi was in a major part due to inadequate technology. With the development of dedicated commercial breast imaging systems, it is believed that there may well be additional radiopharmaceuticals that may prove to be better than or complementary to sestamibi in the evaluation of breast pathology. Hence a long term goal is the development of the appropriate technology that will give investigators the tools to fully evaluate the potential clinical and research applications of these radiopharmaceuticals.

TABLE 1
Partial list of potential single photon,
breast imaging radiopharmaceuticals

| Compound | Status |
|---|---|
| Antibodies | |
| Tc-99m HIS₆-(Z_{HER2:4})₂ HER2 Receptor | Experimental |
| Tc-99m Anti-CEA | Approved |
| In-111 satumomab pendetide (Oncoscint) | Approved |
| Peptides | |
| Tc-99m depreotide | Approved |
| In-111 octretide | Approved |
| Tc-99m bombesin | Experimental |
| Chemotherapy | |
| I-123 tamoxifen | Experimental |
| Hormone | |
| I-123 estradiol | Experimental |
| Lipophilic/cationic agents | |
| Tc-99m sestamibi | Approved |
| Tc-99m tetrofosmin | Approved |
| Protein | |
| Tc-99m annexin-V | Approved |
| Factor | |
| In-111 vitamin B12 | Experimental |
| Metabolism/Perfusion | |
| Tc-99m thio-glucose | Experimental |
| Tc-99m glucarate | Experimental |
| Tc-99m EC-deoxyglucose | Experimental |
| Tc-99m exametazine | Approved |
| TI-201 Thallium Chloride | Approved |
| Tc-99m methylene diphosphonate | Approved |
| Tc-99m (v) DMSA | Approved |

In the recent decade there have been many radiopharmaceuticals studied in both preclinical (animal models) as well as clinical trials with breast cancer patient populations. A limited sampling of some of the radiopharmaceuticals that have been studies is shown in Table 1 above. This illustrates the large array of radiopharmaceuticals that have promising characteristics for breast imaging, but which we believe have been poorly studied due to the lack of appropriate technology that can provide high resolution quantitative information. Several of these compounds are already FDA approved and have demonstrated promising results in breast cancer, including TI-201 Thallous chloride, Tc99m Exametazine, Tc-99m (V) DMSA, Tc-99m tetrofosmin and Tc-99m Glucoheptonate. In addition, newer investigational compounds such as Tc-99m Bombesin appear to have even greater tumor uptake than sestamibi.

Apart from tumor detection, compounds such as I-123 tamoxifen offer researchers a valuable tool in understanding this widely used treatment for breast cancer. The clinical effects of tamoxifen with respect to efficacy and toxicity vary widely among individuals. It is now know that many drugs (such as antidepressants) interfere with the action of tamoxifen (ref Deb). The ability to monitor and quantitate the uptake of 1-123 tamoxifen in the breast in response to various drug regimes is just one example of the value of a dedicated breast imaging system in the understanding of various therapies for breast cancer. Radiolabeling of other therapeutic agents will provide clinicians with the ability to predict the likelihood of response to endocrine therapy in patients with breast cancer. All of this work is heavily dependent on technology that can provide high resolution images of the breast and permit accurate quantification of breast activity. In this respect the development of a clinically optimized breast imaging system is a useful element for both clinical practice and research in breast imaging.

In addition to evaluating the sensitivity of these compounds relative to other breast imaging modalities, many of these agents should be revisited with the goal of determining what molecular information each agent can offer. The current proposal involves the development of a flexible, standardized, quantitative tool to allow such renewed efforts to be performed.

We believe that the field of molecular imaging, especially nuclear imaging as in this proposal, has a bright future that involves the monitoring of all of these technologies in vivo. A review of recent literature on scintimammography shows that clinically, today, most physicians will only consider scintimammography as a last resort in difficult-to assess cases. A leap in molecular medicine can circumvent this inertia in breast care and utilize the full potential of molecular imaging in the detection and evaluation of breast cancer.

Preliminary Data

Phantom Studies and Patient Data

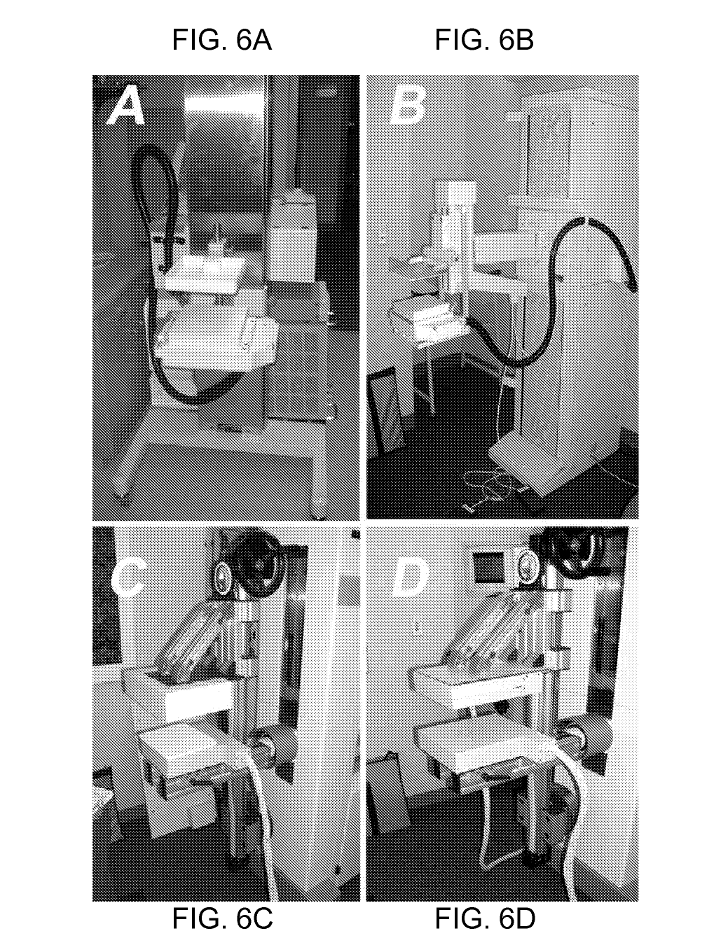

At Mayo Clinic, we have been working on the development of semi-conductor technology for breast imaging since 2001, both in terms of the physics of this technology and in its application in clinical studies. As our experience in this area has grown, it has been reflected in better detectors and gantry designs for breast imaging. FIGS. 6A through 6D show the progression of MBI systems at Mayo since 2001, ranging from the first detector mounted on a modified thyroid uptake probe stand, to today's dual-head detector system incorporated into a modified mammographic gantry.

FIG. 6A shows an original version with prototype GE detector mounted on a modified uptake probe stand. Built in 2001 and used for 3 months. FIG. 6B shows a version 2; same detector as 6A) now mounted on a modified mammographic gantry. Light motorized compression and rotation possible. Constructed in 2002 and used until September 2005. FIG. 6C shows a version 3; first dual-head MBI system, using the original GE CZT detector (upper head) and a Gamma Medica CZT detector on the lower head. Manual breast compression. Constructed in October 2005 and used until March 2006. FIG. 6D shows a version 4; dual head MBI system using 2 matched Gamma Medica CZT detectors. Constructed in March 2006 and in clinical use at present.

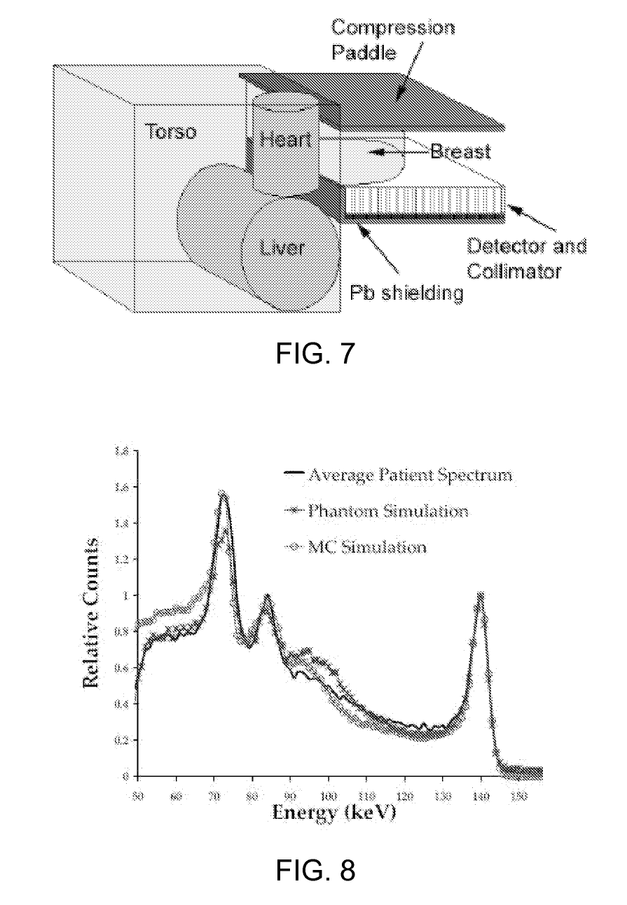

Concurrent with the improvements in gantry design has been a better understanding of the optimal detector characteristics for breast imaging. We have completed both phantom studies and Monte Carlo simulations to evaluate the effects of energy resolution, intrinsic spatial resolution, collimator selection and count density on image quality in breast imaging (refs). FIG. 7 shows a schematic diagram of the experimental phantom model of the patient and detector. With appropriate organ activity, the energy spectra from the experimental model could be matched to that seen in patients. This imaging geometry and appropriate activity was then modeled using MCNP code to simulate the imaging configuration in clinical studies. FIG. 8 shows the energy spectra from the experimental phantom model and the Monte Carlo simulation compared to the average energy spectrum obtained in patients. As can be seen there is excellent agreement between the patient data and both the experimental and computer simulated models. We can decompose the Monte Carlos spectrum and have found that at energy resolutions of 10% or less, <13% of counts in the breast image are scattered events (primarily first order Compton) and scatter from the torso region accounts for less than about 3% of counts in the breast image. At energy resolutions of about 15% and higher there was a significant increase in scatter both within the breast and from the torso (ref). Events from the torso are concentrated at the chest wall edge of the detector's field of view, decreasing tumor detection in this area. Because of low overall scatter in the breast, changes in energy resolution between about 4% and about 10% were found to have minimal effect on tumor detection, even for lesions close to the edge of the detector (ref). Current CZT detectors all achieve an energy resolution of about 7% or less and hence are ideal for high contrast breast imaging.

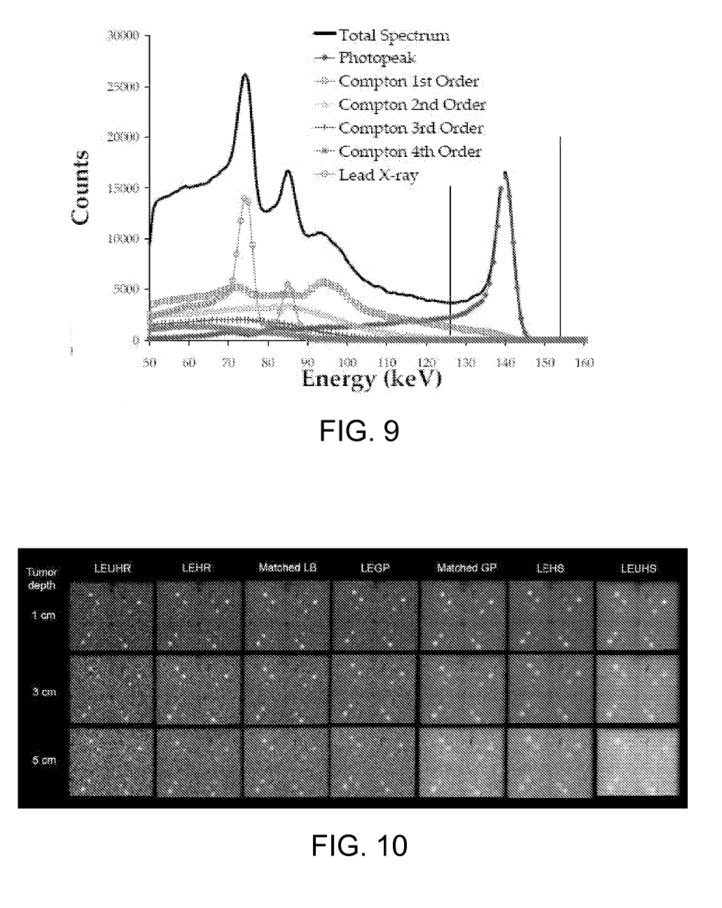

FIG. 9 is a graph showing the energy spectrum from MC simulation showing the contribution of the various components of the spectrum, with the vertical lines showing limits of the standard energy window

The count density in images of the breast acquired with dedicated breast imaging systems is known to be very low. This has a significant effect on the detection of small lesions (e.g., less than about 1 cm) and makes the choice of collimation particularly relevant. We have shown using a range of collimators in three pixilated gamma cameras that a high sensitivity or general purpose collimator gave higher SNR values for about 4-9 mm tumors compared to a high resolution or an ultra-high sensitivity collimator (ref). FIG. 10 shows images of a breast phantom containing tumors about 4-9 mm in diameter and imaged at depth of about 1-5 cm with a tumor/background ratio of about 10:1. All images were acquired on a CZT detector with conventional hexagonal hole collimators (e.g. LEUHR, LEHS, etc) or matched square hole collimators (hole size matched to detector pixel size). LB=long bore, GP=general purpose. The images were acquired on the CZT detector using 7 different types of collimation, ranging from ultra high resolution to ultra high sensitivity.

Optimal collimation was found to be partly dependent on tumor depth, and for tumors within about 3 cm of the collimator face, a low energy all—purpose or high sensitivity collimator was optimum. With a dual-head system and an average breast thickness of 6 cm (ref), tumors can never be more than about 3 cm from the collimator face. Hence collimation was optimized for this distance (ref).

Using optimal detectors and collimation, preliminary results from our laboratory using a dual-head system have demonstrated a very high sensitivity for the detection of small breast tumors. In 38 with confirmed breast cancer, a total of 58 lesions were identified at surgery. Table 2 below presents a breakdown of tumor size, number of tumors and number detected by MBI. Overall sensitivity was about 93%. The sensitivity for tumors less than about 10 mm in size was about 90%. All 4 tumors missed by MBI were IDC. Of these one was about 2 mm in size and below the resolving power of the MBI system and two were missed due to a positioning errors.

TABLE 2
Tumor size and sensitivity of MBI in
the 38 patients with confirmed cancer

| | | | | False | |
|---|---|---|---|---|---|
| Tumor Size | | Total # | True Positive | Negative | Sensitivity (%) |
| <5 | mm | 8 | 7 | 1 | 88 |
| 6-10 | mm | 24 | 22 | 2 | 92 |
| 11-15 | mm | 8 | 7 | 1 | 88 |
| 16-20 | mm | 10 | 10 | 0 | 100 |
| >20 | mm | 8 | 8 | 0 | 100 |

| | | | | |
|---|---|---|---|---|
| All | 58 | 54 | 4 | 93 |

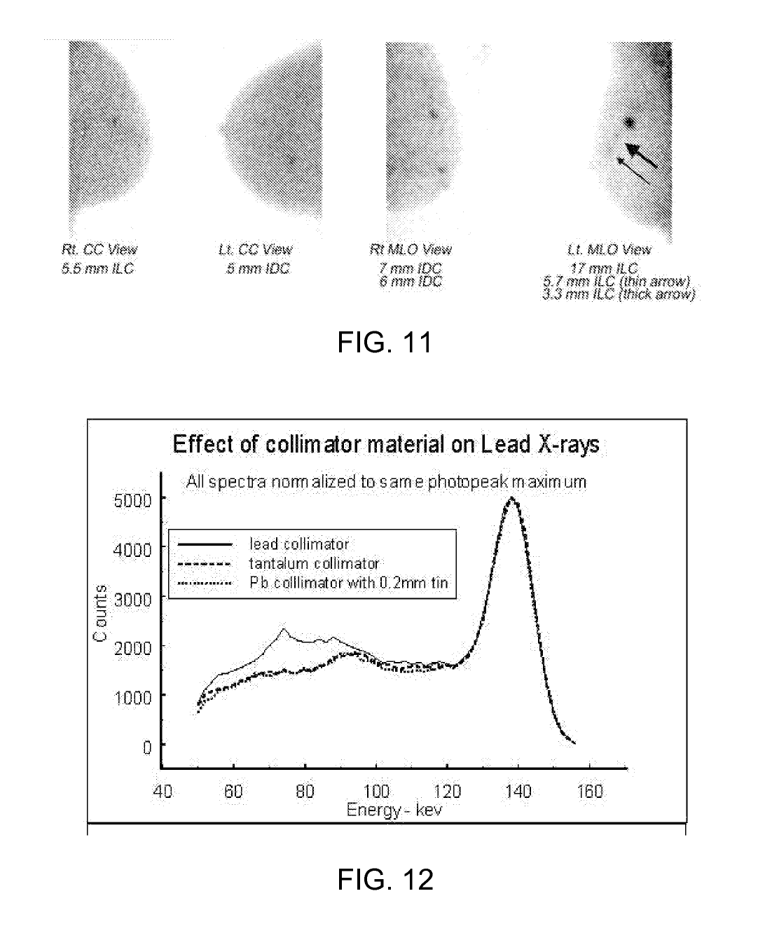

This sensitivity is approximately 15% higher than previously obtained in our laboratory with a single CZT detector system and about 40% higher than that obtained with conventional gamma cameras. FIG. 11 shows examples of breast tumors detected with our dual-head MBI system that range from about 3.3 to 17 mm in diameter. Note the clarity with which lesions as small as about 5-6 mm can be seen. From phantom studies we have confirmed that lesions less than about 10 mm in size are only visible when the relative tumor/background activity is of the order of about 20:1 to about 40:1. The generally accepted tumor/background activity for sestamibi is about 6:1 (ref). Our results would indicate that the true ratio is considerably higher than previously reported, making sestamibi a very attractive radiopharmaceutical for breast cancer, provided one has the appropriate technology to take advantage of this high uptake.

At Mayo Clinic, we are currently using our dual-head system in a comparative study between MBI and screening mammography to screen asymptomatic women at high risk of breast cancer who have mammographically dense breasts. In this study, we have currently scanned about 150 patients (out of a study goal of about 2000 patients). To date, four cancers had been identified by MBI, as well as one pre-cancerous lesion (atypical ductal hyperplasia). Only one of these cancers was identified by mammography and mammography has not detected any cancers that were not seen on MBI. While this is very preliminary data, these early results are very promising and indicate that MBI may be a valuable complementary screening tool to mammography in patients with dense breast tissue on mammogram.

Detector Technology

The current dual-head system employs two Lumagem 3200S detectors (Gamma Medica-Ideas). These are CZT detectors with active areas of about 20×16 cm. Intrinsic spatial resolution is about 1.6 mm and energy resolution is approximately 4%. Both detectors have low energy high sensitivity collimators optimized for breast imaging and hence are ideally suited for MBI. We have performed over 350 MBI studies in our laboratory over the last four years. This experience has provided us with significant intellectual know-how on all aspects of optimum detector characteristics for breast imaging, gantry design, detector separation and placement, shielding requirements, compression techniques and other factors useful to a clinical useful breast imaging system. We believe that with Mayo Clinic's experience with gantry design and patient studies, combined with Gamma Medica-Ideas detector technology and ability to design and fabricate the gantry, we are ideally positioned to create a clinically useful dedicated nuclear breast imaging system.

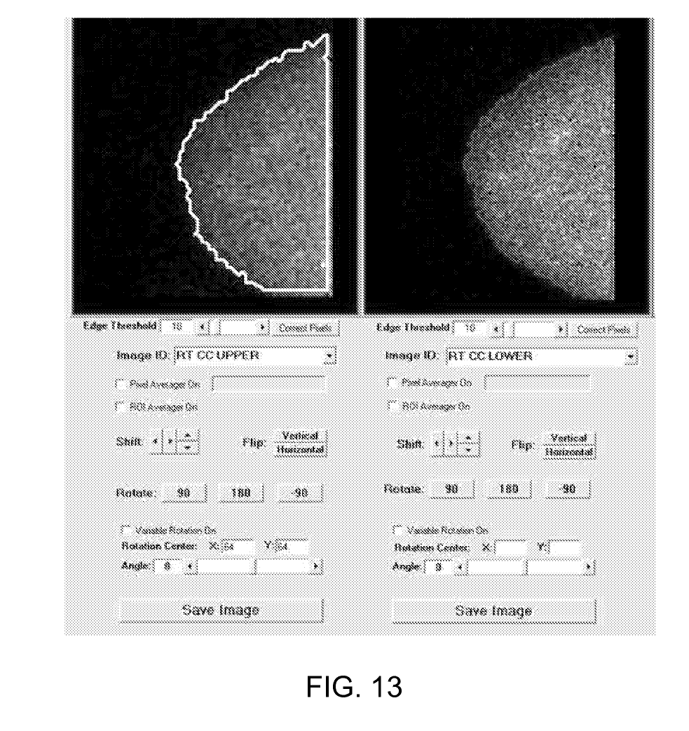

In addition to the above, we are also well positioned to advance the capabilities of MBI into the area of dual-isotope studies, which may be a consideration for researchers evaluating breast physiology. For dual-isotope work with TI-201 and Tc99m, one of the confounding problems is contamination of the TI-201 images with x-ray generated in the lead collimator from the about 140 key photons of Tc-99m. These lead x-rays are highly spatially related to the Tc-99m photopeak image and hence are a significant contaminant in the TI-201 image. We plan to manufacture a foil collimator with a thin layer of tin covering the lead. Monte Carlo simulations performed in our laboratory on such a design indicate that about a 0.2 mm layer of tin coating the collimator holes is capable of absorbing a high percentage of the lead x-rays (FIG. 12). Hence this collimator, combined with the excellent energy resolution of CZT should permit accurate simultaneous Tc-99m/TI-201 imaging for studies evaluating the uptake of Thallous Chloride and Tc-99m or I-123 labeled radiopharmaceuticals in the breast.

Software Development

In the nuclear medicine laboratory, Dr O'Connor directs a team of 3 programmers who have developed a suite of image display and analysis programs called Mayo Image Studio. These programs have been developed over the last 7 years and run on standard Windows NT or XP workstations. The software uses DICOM as its internal file format and can also read in Interfile file format. The software permits a large variety of image display and analysis functions to be performed. It also allows the user to export curves generated as part of an analysis in a .csv format that can be read by Excel and other similar types of spreadsheets. This software permits investigators to access nuclear medicine, PET, CT and MRI images on their desktop workstations and reduces dependence on expensive proprietary image analysis workstations. There are over 50 workstations in the laboratory running this software.

Currently, the Mayo Image Studio contains 4 display programs (orientated towards different types of data—nuclear medicine and MRI), and about 50 different analysis programs ranging from software for general image manipulation to specific programs for analysis of, for example, measurement of I-131 distribution in the body. Software is written in Visual Basic and uses an Active X component (Mayo Active X) that provides basic display and analysis capabilities. Software development time is typically an order of magnitude faster than conventional software methods. Using these software tools, preliminary work is already underway to develop software for alignment of images from opposing heads. This software will be used to generate geometric images of the breast as well as correct for image acquisition problems common to CZT detectors (hot pixels, dead pixels, image misalignment, incorrect orientation, incorrect labeling etc.). FIG. 13 shows the prototype user interface for this program.

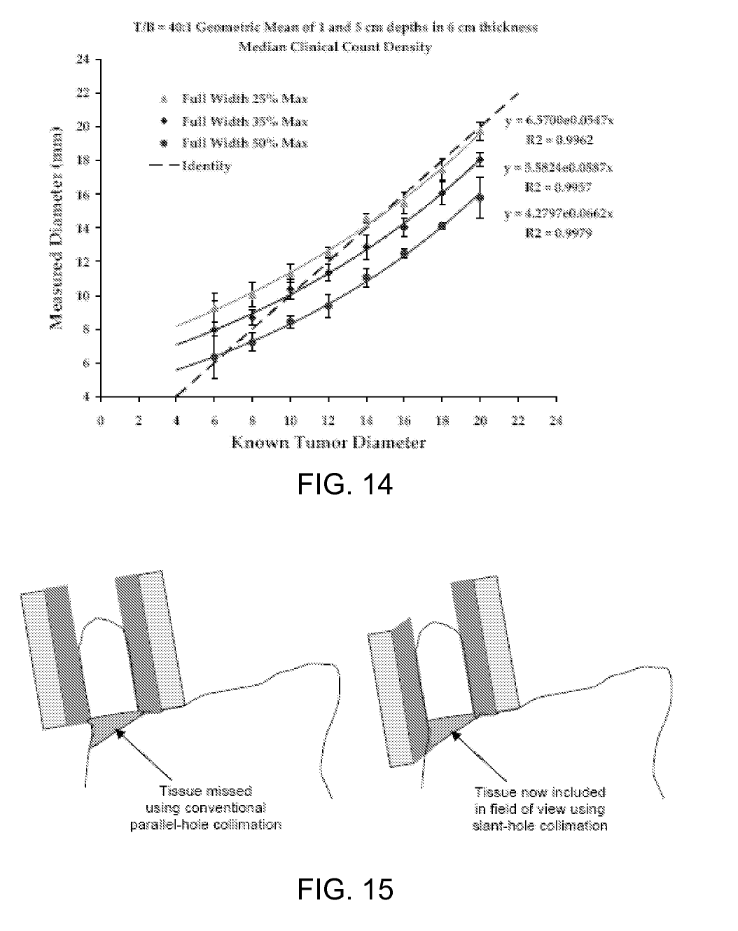

A second program will be written to permit quantitation of tumor depth, size and tumor/background ratio. The algorithms for these calculations will be derived from Monte Carlo simulations of breast tumors. Because of the use of matched opposing detectors, we can generate geometric mean (GM) images that effectively place suspected lesions in the middle of a breast of known thickness (simply the separation of the detector heads). From the geometric image we can estimate tumor diameter. As an example of how this will be achieved, FIG. 14 shows the correlation between true and measured tumor diameter using various percentages of the width of the tumor profile. Analysis was performed from the geometric mean of opposing images. The images used in this analysis were generated at about a 40:1 tumor to background ratio and at median count density seen in clinical studies. This process would be repeated for different tumor depths, breast thicknesses, tumor/background ratios, etc. From knowledge of tumor depth, tumor diameter and tumor counts we can accurately assess true tumor/background ratio and assuming a uniform attenuation coefficient for breast tissue, we can obtain an estimate of absolute tumor activity. Thus this software combined with a dual-detector system will permit quantitation of lesion uptake of radiopharmaceuticals and can permit better discrimination between benign and malignant processes, as well as provide researchers with an ideal tool for quantitative molecular imaging of the breast.

Exemplary Research Design and Methods

Phase 1 Specific Aim 1: Construction of a Molecular Breast Imaging system Hypothesis: MBI is a very promising technique for the detection of breast cancer. The availability of an imaging system optimized for routine clinical use is useful for the widespread adoption of this technology.

Rationale: We have demonstrated that a dual-head system using state-of-the-art detectors from Gamma Medica-Ideas, can achieve about 90% sensitivity for the detection of about sub 10 mm lesions in the breast. For studies performed to date, the gantry and mounting for the detectors have been manufactured at Mayo and our five years of experience in this field has enabled us to optimize the construction of a working breast imaging system. Our results have yet to be reproduced in other laboratories, as many institutions lack the technical know-how and facilities to create such a system. Hence a component of this project is the construction by Gamma Medica-Ideas of fully functional prototype Molecular Breast Imaging systems. At the end of Phase 1, prototypes will be placed in Mayo clinic, Cedars Sinai Medical Center (Los Angeles, Calif.) and in Memorial Sloan-Kettering Medical Center (New York, N.Y.). In Phase II, both Cedars Sinai Medical center and Memorial Sloan-Kettering Medical Center will perform clinical studies to validate results obtained at Mayo Clinic.

Experimental Design: Gamma Medica-Ideas will manufacture a dual-detector MBI system. This would include the following design parameters:
1. Dual-head, opposing CZT detectors, each with about a 6″×8″ field of view (about 15.24 cm×20.32 cm);
2. Opposing detectors are aligned with adequate separation between heads for positioning the breast (minimum of about 20 cm);
3. Gantry will have the ability to acquire cranio-caudal, medio-lateral, and axillary views of the breast;
4. Automated light compression with manual override and digital readout of compression force;
5. Automated recording of breast thickness for each view—result stored in image header;
6. System will accommodate an optional slant hole collimator for better visualization of breast tissue close to the chest wall. This may require that detector heads have the ability to slide relative to each other while maintaining constant separation;
7. Rapid collimator exchange on lower detector to accommodate item 6 above;
8. Optimization of slant-hole collimation for lower detector (see below); or
9. Single computer controlling data acquisition from both detectors.

Design Milestones. Progress toward the accomplishment of the first specific aim, namely the design and assembly of the dual-head CZT-based MBI system, will be measured by comparison with the following milestones:
1. Gantry Design Complete
2. Detector Arm Design Complete
3. Detector Housing Complete
4. Data Acquisition Subsystem Design Complete
5. Software Interface Design for Dual-head Complete

Gantry Fabrication Milestones
1. Gantry Fabrication complete
2. Arm Fabrication complete
3. Detector housings complete
4. Compression device complete
5. Data Acquisition subsystem complete

MBI System Assembly Milestones
1. Gantry and arm assembled and tested
2. Arm and detector housings and compression device assembled and tested
3. Electronics connected with power supplies and acquisition tested
4. Image acquisition software functional and tested
5. Dual-head images acquired with heads assembled in gantry

Although, this list of tasks and the checklist of milestones appears to be overly ambitious to accomplish within the time period estimated in this project plan. Therefore, the uncharted design territory includes:
1. Sliding detectors (and the software corrections for image registration)
2. Design and fabrication of rotatable mammography arm
3. Design and fabrication of adjustable-separation CZT detector heads

The remainder of the tasks, including the dual head acquisition, have been accomplished and documented for other product development projects. For example, the acquisition of dual-head images is routinely performed on the Gamma Medica-Ideas X-SPECT product for small animal imaging.

Rationale for Slant Hole Collimator and sliding detector apparatus. FIG. 15 shows the main reason for the fabrication and testing of a slant hole collimator in the dual-head imaging device. When a tumor is located near the muscular tissue near the chest wall, the dual head system may not “see” the tumor in its combined field of view. Shifting the lateral detector forward under the patient's arm permits visualization of breast tissue close to the chest wall. However, it may also contaminate the breast images with activity from the myocardium. With conventional parallel hole collimation, it is often difficult to include the medial superior aspects of the breast in the detector field of view. The use of a slant hole collimator allows the tumor near the chest wall to be visualized by the lateral detector without contamination from activity outside the breast.

Phase 1 Specific Aim 2: Development of software for display and analysis of breast images Hypothesis: Coupled with the construction of the MBI system, it is useful to also develop the software that can take advantage of the useful information present in these images.

Rationale: Clinical experience at Mayo Clinic has demonstrated the utility in developing a suite of software programs that can a) display the images in a format comparable to conventional mammography, b) correct for labeling, rotation, sizing, and alignment errors in the original images when required, and c) extract quantitative information on tumor depth, diameter and uptake. At the end of Phase 1, copies of this software will be distributed with the prototype clinical systems to Cedars Sinai Medical Center (Los Angeles, Calif.) and in Memorial Sloan-Kettering Medical Center (New York, N.Y.).

Experimental Design: Gamma Medica-Ideas and Mayo Clinic will develop the software for image display and analysis. This would include the following design parameters:

Computer software to perform the following functions:
a. display program to present images in standard mammography views;
b. all images to be stored in DICOM format with capability of being exported to digital mammography DICOM PACS display and analysis system;
c. alignment of opposing views and generation of geometric mean image;
d. ability to adjust for mis-alignment between detector heads;
e. quantification of tumor diameter and depth; or
f. quantification of true T/B ratio, adjusted for tumor size

Rendering of a single Geometric Mean image

The dual head MBI system will provide two opposing images of the breast. When the detector heads are exactly opposite each other, they will provide the same view of the breast tissue. Hence a standard geometric mean image can be generated that will provide a single view of the breast and mathematically place lesion activity in the middle of the breast. With knowledge of breast thickness, the distance of the lesion from either detector is simply half the breast thickness.

Quantitation of Tumor Size and Depth

Knowledge of breast thickness and the attenuation coefficient for soft tissue will allow estimation of tumor depth from the ratio of counts in opposing view. Tumor size will be estimated from the Geometric Mean (GM) image of the breast. Count profiles through breast lesions will be taken at multiple projections. Estimation of tumor diameter will be performed by calculating the profile width at various percentages of peak counts. The profile full width at about 3 percentages of the maximum profile counts (about: 50%, 35% and 25%) will be input into a look-up table to estimate tumor diameter. Using multiple thresholds has been found to provide a more reliable estimate of tumor diameter in low count data. Tumor diameter will be estimated in both X and Y directions and an average tumor diameter in X and Y direction will be obtained. Analysis will be done on both the craniocaudal and mediolateral oblique views to enable a more accurate estimate of tumor size. A series of look-up tables will be generated from Monte Carlo simulations of the gamma camera and collimator along with simulated breast images of various thickness and containing tumors of various sizes.

Measurement of Tumor/Background Ratio

Knowledge of tumor size is useful to accurate estimation of tumor/background (T/B) ratio. From the estimated tumor volume, counts from an equal volume of surrounding background tissue will be obtained to yield a T/B ratio. Validation of results will be performed using compressible breast phantom models. A compressible breast phantom will be constructed using a gelatin core and a latex outer skin (34). The gelatin will be mixed with water and Tc-99m to create the appropriate background activity. Small lesions (less than about 1 cm in diameter) will be simulated using wax encapsulated gelatin spheres containing Tc-99m at a various concentrations relative to the background activity and embedded within the breast phantom. Images of this phantom will be used to develop and validate the software for estimation of the true T/B ratio.

DICOM Format

Our research group will become familiar with the standards for digital display and archival of mammographic images as formatted in DICOM. Digital mammography by definition is a computer rendering standard. We will learn about the digital mammographic DICOM format and render our dual-head images into this format for export to PACS systems that can display our data alongside the corresponding digital mammograms.

Advanced Image Processing

There are several techniques that are known and emerging in the scientific literature that might potentially improve the dual-head image quality, reduce the scan time, or render the lesions more visible. Once the MBI systems are operational and producing reliable and consistent clinical results, we will turn out attention to these issues. These include:
a. Adaptive smoothing—this will reduce image noise and may potentially reduce scan time or improve lesion detection
b. Resolution recovery—measured differences in the same lesion provides a first estimate to the lesions position and size. An iterative reconstruction technique can then be applied to optimize the GM images while maintaining consistency with the original two planar images.
c. Use of the slant hole's about 30-degree perspective to improve the estimate of tumor depth.

Time Line for Phase I Work

A working prototype of the proposed system has already been assembled at the Mayo Clinic. Since it was a “field upgrade”, no part of the gantry or the detector support system was initially designed to hold two CZT detectors. Additionally, it does not have the capability of sliding the detectors relative to each other as required for work with slant-hole collimators. The research plan described above details how the Mayo prototype will be redesigned to be a next-generation dual-head Molecular Breast Imaging tool, complete with the software for quantitative analysis of the images.

Obviously the cost of a dual head camera is considerably more than a single head when dealing with semiconductor such as CZT. There will be redundancies in power supplies and acquisition boards that avoid the replication of the complete single-head system, but the CZT detector modules are the most costly component in the proposed system and we will look for ways to reduce this cost and make the product more commercially viable. One way that will be investigated is to analyze the usefulness of the “corner” modules in the rectangular field of view—Since the breast tends to retain its conical or triangular shape even with light compression, few counts are obtained in the corner modules furthest from the patient. We will evaluation the possibility of removing these modules from the design from a cost-cutting perspective.

Phase 2 Specific Aim 1: Validation of dual detector MBI system Hypothesis: Preliminary results from Mayo Clinic indicate that MBI is a very promising technique for the detection of breast cancer. Validation of the results obtained at Mayo is useful. Feasibility studies can to be performed to further explore the potential applications for this technology in the future.

Rationale: Limited clinical studies at Mayo Clinic have demonstrated the potential value of a dual-headed MBI system in the detection of small breast tumors. However, it is desirable that the preliminary results from Mayo Clinic be confirmed in other laboratories. Prototype clinical systems in Cedars Sinai Medical Center (CSMC) and in Memorial Sloan-Kettering Cancer Center (MSKCC) will be used to confirm the results obtained at Mayo Clinic. In addition feasibility studies will be performed to further evaluate potential applications for this technology.

Exemplary Experimental Design: A total of about 200 patients will be studied in CSMC and MSKCC. Each patient will have a suspicious lesion on mammogram for which biopsy is scheduled. The following inclusion criteria can be applied:
Lesion size on mammogram be about 2 cm or less in diameter;
Lesions be BIRAD category of about 4-5 (“suspicious” or “highly suspicious of malignancy”);
Patient be 18 years of age or older; or
Negative pregnancy test or postmenopausal or surgically sterilized.

Patients with prior needle biopsy of the lesion will be excluded from this study; as such biopsies may effectively remove all or part of the lesion. All patients will undergo cranio caudal (CC) and medio-lateral oblique (MLO) views of each breast with the dual-head MBI system. Images will be processed and displayed for analysis using the MBI software developed in Phase 1. An estimation of the sensitivity of the dual-head MBI system for the detection of breast lesions as a function of lesion size will be determined. Absolute T/B ratio will be determined to see if it provides additional diagnostic information on the nature of a lesion (benign/malignant).

Phase 2 Specific Aim 2: Comparison of MBI and MRI Hypothesis: MBI will be a cost-effective alternative to breast MRI in many of the patient populations currently referred to MRI for additional evaluation.

Rationale: In over 20 patients who have undergone both MBI and MR studies of the breast, remarkable concordance has been observed in image sets from both modalities. Hence this project will expand on this finding to encompass the full range of indications used for MR studies. In order to obtain sufficient data, studies will be performed at the three centers and the results read by blinded observers.

Experimental Design: A total of about 300 patients will be studied in CSMC, MSKCC and Mayo Clinic. All patients will be scheduled for an MR study of the breast, either for screening or for further evaluation of indeterminate mammographic or ultrasound procedures. The following groups will be evaluated:
Follow-up in patients post-radiation therapy
Evaluation of patients with indeterminate findings on mammogram or ultrasound
Screening of patients with the BRCA 1 or 2 gene mutation

All patients will be scheduled for a clinical MR study of the breast. Patients will be excluded from this study if any interventional procedure is performed between the time of the MR and MBI studies, as such procedures may effectively remove all or part of the lesion or result in false positive uptake at the site of the intervention. All patients will undergo cranio caudal (CC) and medio-lateral oblique (MLO) views of each breast with the dual-head MBI system. Images will be processed and displayed as described above for Aim 1.

For analysis, data will be sub-divided into the 2 groups defined above. Using final pathology (for malignant lesions) or needle biopsy results (for benign conditions) as the gold standard the relative sensitivity of the two modalities will be evaluated.

Phase 2 Specific Aim 3: Feasibility of TI-201 Thallous Chloride Imaging Hypothesis: All previous work with CZT detectors has focused on imaging the about 140 keV gamma radiation from Tc-99m. When the energy spectra for TI-201 acquired on a conventional gamma camera and on a CZT detector are compared, the improved energy resolution achieved at low energies with TI-201 can result in a improvement in images with this radiopharmaceutical. Monte Carlo simulations of the breast and detector will demonstrate the relative quality of TI-201 and Tc-99m based images and will permit derivation of the appropriate algorithms for estimation of tumor size and uptake.

Rationale: While Tc-99m sestamibi has demonstrated excellent uptake in malignant breast conditions, it also shows high uptake in several benign conditions such as fibroadenoma, fat necrosis, inflammation etc. In addition, in some patients rapid washout can occur making lesion detection difficult. Thallium chloride is known to have excellent uptake in breast tumors, but image quality is usually degraded due to the high scatter content in thallium images and the sub-optimal imaging characteristics of conventional gamma cameras at low energies. CZT detectors retain excellent energy resolution at lower energies and this can allow use of narrowed energy windows and improve image contrast. The objectives of this study are to model the energy spectrum acquired during planar clinical imaging of the compressed breast with TI-201, to quantify the fraction of scattered events that occur in the energy-windowed image, and to evaluate the effects of changes in energy resolution and energy window on scatter in the image and lesion detection.

Exemplary Experimental Design: Monte Carlo simulations using MCNP code will be used to simulate detector geometry. We will model the LumaGem 3200s system, which comprised about a 96×128 array of cadmium zinc telluride (CZT) elements with about 1.6 mm-pixels and high sensitivity collimator. The patient model will consisted of an 800-mL breast compressed to a thickness of about 5.5 cm and an adjacent about 8000-mL torso containing compartments modeling the liver and heart. Energy spectra from 5 female patients undergoing TI-201 myocardial perfusion scans will be acquired to determine an average patient energy spectrum. A phantom simulation will be performed to determine the activity concentration in liver and heart regions vs. the torso cavity and breast that produced an energy spectrum most closely matching the average patient spectrum.

Intrinsic energy resolution of the detector will be determined experimentally and entered into the model. A correction to model the tailing effect in the CZT will be included. An image of 3 tumors, each about 1 cm in diameter, placed at various distances from the chest wall, will be simulated by creating an image of photons captured in the CZT with energies within energy windows of various widths. The spectral components and their contribution to the energy windowed image will be examined and the effect of changes in energy window on tumor detection will be determined.

Once the optimal energy settings have been established, simulations will be run for various breast thicknesses, tumor diameters and tumor/background uptakes to permit development of the appropriate algorithms for calculation of tumor size and tumor/background uptake.

Phase 2 Specific Aim 4: Clinical Evaluation of Thallous Chloride Hypothesis: TI-201 thallous chloride has been used for many years for tumor imaging. With more optimal imaging technology that eliminates much of the scatter component present in images acquired with conventional gamma cameras, this radiopharmaceutical will show equal or better uptake in breast tumors than Tc-99m sestamibi.

Rationale: While Tc-99m sestamibi has demonstrated excellent uptake in malignant breast conditions, it also shows high uptake in several benign conditions such as fibroadenoma, fat necrosis, inflammation etc. In addition, in some patients rapid washout can occur making lesion detection difficult. Thallium chloride is known to have excellent uptake in breast tumors, but image quality is usually degraded due to the high scatter content in thallium images and the sub-optimal imaging characteristics of conventional gamma cameras at low energies. CZT detectors retain excellent energy resolution at lower energies and this can allow use of narrowed energy windows and improve image contrast.

Exemplary Experimental Design: Following successful conclusion of Specific Aim 3 above, a total of about 100 patients will be studied. Patients will be randomized to receive either Thallium chloride or Tc-99m sestamibi. Average uptake in each group will be used to compare the different radiopharmaceuticals. Each patient will have a suspicious lesion on mammogram for which biopsy is scheduled. The following inclusion criteria can be applied:
Lesion size on mammogram be about 4 cm or less in diameter;
Lesions be BRAD category of about 4-5 (“suspicious” or “highly suspicious of malignancy”);
Patient be 18 years of age or older; or
Negative pregnancy test or be postmenopausal or surgically sterilized.

Patients with prior needle biopsy of the lesion will be excluded from this study, as such biopsies may effectively remove all or part of the lesion. All patients will undergo cranio caudal (CC) and medio-lateral oblique (MLO) views of each breast with the dual-head MBI system. Images will be processed and displayed for analysis as described in Aim 1. The absolute tumor/background uptake ratio in will be determined and categorized by tumor type. Studies will be performed at both CSMC and MSKCC. No significant restriction of lesion size will be in place as the purpose of this study is the evaluation of the relative uptake of the two radiopharmaceuticals.

Phase 2 Specific Aim 5: Evaluation of Alternative Radiopharmaceuticals Hypothesis: Alternative radiopharmaceuticals to Tc-99m sestamibi and Thallium Chloride may prove to have more attractive characteristics as tumor imaging agents.

Rationale: While Tc-99m sestamibi has demonstrated excellent uptake in malignant breast conditions, it also shows high uptake in several benign conditions such as fibroadenoma, fat necrosis, inflammation etc. In addition, it delivers a moderate radiation dose to the bowel and stomach. Alternative radiopharmaceuticals will be evaluated to see if any offer better imaging characteristics to Tc-99m sestamibi, either with respect to dosimetry, tumor uptake or reduced uptake in benign conditions.

Exemplary Experimental Design: A total of 5 alternative radiopharmaceuticals to Tc-99m sestamibi will be evaluated. For each radiopharmaceutical, a total of about 50 patients will be studied. Since it will not be practicable to evaluate more than one radiopharmaceutical in each patient, population averages will be used to compare the different radiopharmaceuticals. Each patient will have a suspicious lesion on mammogram for which biopsy is scheduled. The following inclusion criteria can be applied:
Lesion size on mammogram be about 4 cm or less in diameter;
Significant cluster of calcifications indicative of DCIS;
Lesions be BIRAD category of about 4-5 (“suspicious” or “highly suspicious of malignancy”);
Patient be 18 years of age or older; or
Negative pregnancy test or be postmenopausal or surgically sterilized.

Patients with prior needle biopsy of the lesion will be excluded from this study, as such biopsies may effectively remove all or part of the lesion. All patients will undergo cranio caudal (CC) and medio-lateral oblique (MLO) views of each breast with the dual-head MBI system. Images will be processed and displayed for analysis as described in Aim 1. For each radiopharmaceutical, the absolute tumor/background uptake ratio in will be determined and categorized by tumor type. In addition to Tc-99m sestamibi, the following radiopharmaceuticals will be evaluated:
Tc-99m tetrofosmin
Tc-99m Glucarate
Tc-99m thio-glucose
Tc-99m bombesin
Tc-99m vitamin B12

As with TI-201 studies, no significant restriction of lesion size will be in place as the purpose of this study is evaluation of relative uptake of the different radiopharmaceuticals.

Phase 2 Other Specific Aims

Other Potential Add-On Projects
1. specific project to evaluate value of slant-hole collimator for improved detection of lesions close to the chest wall
2. evaluation of ductal carcinoma in-site (Above projects all focus on patients with lesions of a defined size)

Therefore, the present invention provides a method for performing quantitative tumor analysis using information acquired with a dual-headed molecular breast imaging system. Specifically, the present invention provides a method for accurately determining the size, depth to the collimator, and relative tracer uptake of a tumor. While determination of these parameters was previously only possible with tomographic imaging methods, the present invention is able to utilize the information available in planar dedicated breast imaging to provide these previously unavailable information sets to aid in the diagnosis and biopsy of the site.

The present invention has been described in terms of one or more preferred embodiments, and it should be appreciated that many equivalents, alternatives, variations, and modifications, aside from those expressly stated, are possible and within the scope of the invention.

### REFERENCES

1. American Cancer Society, 2005. Cancer Facts and FIGS. 2005 (available at: http://www.cancer.orq/downloads/STT/CAFF2005f4PWSecured.pdf).

2. Nystrom L, Andersoon I, Bjurstam N. Long-term effects of mammography screening: updated overview of the Swedish randomized trials. Lancet 2002; 359:909-19.

3. Fletcher S W, Black W, Harris R, Rimer B K, Shapiro S. Report of the International Workshop on Screening for Breast Cancer. J Natl Cancer Inst 1993; 85:1644-1656.

4. Hendrick R E, Smith R A, Rutledge J H, Smart C R. Benefit of screening mammography in women aged 40-49: a new meta-analysis of randomized controlled trials. J Natl Cancer Inst Monographs 1997; 22:87-92.

5. U.S. Preventive Services Task Force. Screening for breast cancer: Recommendations and rationale. Ann Intern Med 2002; 137:344-346.

6. Mann B D, Guiliano A E, Bassett L W, et al. Delayed diagnosis of breast cancer as a result of normal mammograms. Arch Surg 1983; 118; 23-25.

7. Carney P A, Miglioretti D L, Yankaskas B C et al. Individual and combined effects of age, breast density, and hormone replacement use on the accuracy of screening mammography. Ann Intern Med 2003; 138:168-175.

8. Ma L, Fishell E, Wright B, Hanna W, Allan S, Boyd N F. Case-control study of factors associated with failure to detect breast cancer by mammography. J Natl Cancer Inst 1992; 84:781-785.

9. Bird R E, Wallace T W, Yankaskas B C. Analysis of cancers missed at screening mammography. Radiology 1992; 184:613-617.

10. Holland R, Hendriks J H, Mravunac M. Mammographically occult breast cancer: a pathologic and radiologic study. Cancer 1983; 52:1810-1819.

11. Kolb T M, Lichy J, Newhouse J H. Comparison of the performance of screening mammography, physical examination, and breast ultrasound and evaluation of factors that influence them: An analysis of 27,825 patient evaluations. Radiology 2002; 225:165-175.

12. Blane C E, Fitzgerald J T, Gruppen L D, Oh M S, Helvie M A, Andersson I. Decreasing rate of fatty involution at screening mammography. Acad Radiol 2002; (8):895-898.

13. Verbeek A L M. The influence of breast density on the sensitivity of mammography screening. Eur J Cancer 2004; 40: Suppl 2, 57.

14. Wolfe J N. Risk for breast cancer development determined by mammographic parenchymal pattern. Cancer 1976; 37:2386-2492.

15. Harvey J A, Bovbjerg V E. Quantitative assessment of mammographic breast density: relationship with breast cancer risk. Radiology 2004; 230:29-41.

16. Boyd N F, Dite G S, Stone J et al. Heritability of mammographic density, a risk factor for breast cancer. N Engl J Med 2002; 347:886-94.

17. Harvey J A, Bovbjerg V E. Quantitative assessment of mammographic breast density: relationship with breast cancer risk. Radiology 2004; 230:29-41.

18. Buist D S M, Porter P L, Lehman C, Taplin S H, White E. Factors contributing to mammography failure in women aged 40-49 years. J Natl Cancer Inst 2004; 96:14321440.

19. Porter P L, El-Bastawissi A Y, Mandelson M T, Lind M G, Khalid N, Watney E A, et al. Breast tumor characteristics as predictors of mammographic detection: comparison of interval and screen-detected cancers. J Natl Cancer Inst 1999; 91:2020-2028.

20. Gilliland F D, Joste N, Stauber, P M et al. Biologic characteristics of interval and screen-detected breast cancers. J Natl Cancer Inst 2000; 92:743-749.

21. Roubidoux M A, Bailey J E, Wray L A, Helvie M A. Invasive cancers detected after breast cancer screening yielded a negative result: Relationship of mammographic density to tumor prognostic factors. Radiology 2004; 230:42-48.

22. Lewin J, Hendrick R E, D'Orsi C J et al, Comparison of Full-Field Digital Mammography with Screen-Film Mammography for Cancer Detection: Results of 4,945 Paired Examinations, Radiology 2001 March Vol 218, 873-880.

23. Cole E B, Pisano E D, Kistner E O, Muller K E, Brown M E, Feig S A, Jong R A, Maidment A D, Staiger M J, Kuzmiak C M, Freimanis R I, Lesko N, Rosen E L, Walsh R, Williford M, Braeuning M P. Diagnostic accuracy of digital mammography in patients with dense breasts who underwent problem-solving mammography: effects of image processing and lesion type. Radiology. 2003 January; 226(1):153-60.

24. Pisano E D, Gatsonis C, Hendrick E, Yaffe M, Baum J K, Acharyya S, et al. Diagnostic Performance of Digital versus Film Mammography for Breast-Cancer Screening. New England J Med 2005: 353: 1773-1783.

25. Kuhl C K, Schmutzler R K, Leutner C C et al. Breast MR imaging screening in 192 women proved or suspected to be carriers of a breast cancer susceptibility gene: preliminary results. Radiology. 2000; 215:267-279.

26. Tilanus-Linthorst M M, Obdeijn I M, Bartels K C, deKoning H J, Oudkerk M. First experiences in screening women at high risk for breast cancer with MR imaging. Breast Cancer Rest Treat. 2000; 63:53-60.

27. Podo F, Sardanelli F, Canese R et al. The Italian multi-centre project on evaluation of MRI and other imaging modalities in early detection of breast cancer in subjects at high genetic risk. J Exp Clin Cancer Res 2002; 21(3 duppl):115-124.

28. Kriege M, Brekelmans C T, Boetes C et al. Efficacy of MRI and mammography for breast cancer screening in women with a familial or genetic predisposition. N Engl J Med 2004; 351:427-437.

29. Warner E, Plewes D B, Hill K A, et al. Surveillance of BRCA1 and BRCA2 mutation carriers with magnetic resonance imaging, ultrasound, mammography, and clinical breast examination. JAMA 2004; 292:1317-132.

30. 30 National Cancer Institute Clinical Trial Results. MRI vs. Mammography: Which is Better for Screening Women at High Risk of Breast Cancer? (http://www.cancer.gov/clinicaltrials/results/mri-vs-mammography0603).

31. Brem R F, Hoffmeister J W, Rapelyea J A, Zisman G, Mohtashemi K, Jindal G, Disimio M P, Rogers S K. Impact of breast density on computer-aided detection for breast cancer. Am J Roentgenol. 2005; 184:439-44.

32. Brem R F, Rapelyea J A, Zisman G, Hoffmeister J W, Desimio M P. Evaluation of breast cancer with a computer-aided detection system by mammographic appearance and histopathology. Cancer. 2005; 104:931-5.

33. Ho W T, Lam P W. Clinical performance of computer-assisted detection (CAD system in detecting carcinoma in breasts of different densities. Clin Radiol. 2003; 58:133-6.

34. Rice S J, Khalkhali I, Diggles L. The role of radionuclide breast imaging in the evaluation of suspected breast abnormalities. Breast Dis. 1998; 10:177-87.

35. Buscombe J R, Cwikla J B, Thakrar D S, Nilson A J. Scintigraphic imaging of breast cancer: a review. Nucl Med Commun. 1997; 18:698-709.

36. Bombardieri E, Crippa F. PET imaging in breast cancer. Q J Nucl Med. 2001; 45:245-56.

37. Reske S N, Kotzerke J. FDG-PET for clinical use. Results of the 3rd German Interdisciplinary Consensus Conference, “Onko-PET III”, 21 Jul. and 19 Sep. 2000. Eur J Nucl Med. 2001; 28:1707-23.

38. Nakamura S, Kenjo H, Nishio T, Kazama T, Do O, Suzuki K. 3D-MR mammography-guided breast conserving surgery after neoadjuvant chemotherapy: clinical results and future perspectives with reference to FDG-PET. Breast Cancer. 2001; 8:351-4.

39. Abreu M C, Almeida P, Balau F, Ferreira N C, Fetal S, Fraga F, Martins M, Matela N, Moura R, Ortigao C, Peralta L, Rato P, Ribeiro R, Rodrigues P, Santos Al, Trindade A, Varela J. Clear-PEM: a dedicated PET camera for improved breast cancer detection. Radiat Prot Dosimetry. 2005; 116:208-10.

40. Murthy K, Aznar M, Thompson C J, Loutfi A, Lisbona R, Gagnon J H. Results of preliminary clinical trials of the positron emission mammography system PEM-I: a dedicated breast imaging system producing glucose metabolic images using FDG. J Nucl Med. 2000; 41:1851-8.

41. Tafra L, Cheng Z, Uddo J, Lobrano M B, Stein W, Berg W A, Levine E, Weinberg I N, Narayanan D, Ross E, Beylin D, Yarnall S, Keen R, Sawyer K, Van Geffen J, Freimanis R L, Staab E, Adler L P, Lovelace J, Shen P, Stewart J, Dolinsky S. Pilot clinical trial of 18F-fluorodeoxyglucose positron-emission mammography in the surgical management of breast cancer. Am J Surg. 2005; 190:628-32.

42. Wu D, Gambhir S S. Positron emission tomography in diagnosis and management of invasive breast cancer: current status and future perspectives. Clin Breast Cancer. 2003; 4 Suppl 1:S55-63.

43. Maublant J C, Zhang Z, Rapp M, Ollier M, Michelot J, Veyre A. In vitro uptake of technetium-99m-teboroxime in carcinoma cell lines and normal cells: comparison with technetium-99m-sestamibi and thallium-201. J Nucl Med. 1993; 34:1949-52.

44. Carvalho P A, Chiu M L, Kronauge J F, Kawamura M, Jones A G, Holman B L, Piwnica-Worms D. Subcellular distribution and analysis of technetium-99m-MIBI in isolated perfused rat hearts. J Nucl Med. 1992; 33:1516-22.

45. Khalkhali I and Vargas H I. The role of nuclear medicine in breast cancer detection: Functional breast imaging. Radiologic Clinics of North America 2001; 39: 1053-1068.

46. Khalkhali I, Villanueva-Meyer J, Edell J L, et al. Diagnostic accuracy of 99mTc sestamibi breast imaging: Multicenter trial results. J Nucl Med 2000; 41: 1973-1979.

47. Palmedo H, Biersack HJ, Lastoria S, et al. Scintimammography with technetium99m methoxyisobutylisonitrile: results of a prospective European multicentre trial. Eur J Nucl Med 1997; 25: 375-385.

48. Tofani A, Sciuto R, Semprebene A, et al. 99Tcm-MIBI scintimammography in 300 consecutive patients: Factors that may affect accuracy. Nucl Med Commun 1999; 20: 1113-1121.

49. Spanu A, Schillaci O, Meloni G B, et al. The usefulness of 99mTc tetrofosmin SPECT scintimammography in the detection of small size primary breast carcinomas. Internat J Oncol 2002; 21: 831-840.

50. Scopinaro F, Pani R, De Vincentis G, Soluri A, Pellegrini R, Porfini L M. High-resolution scintimammography improves the accuracy of technetium-99m methoxyisobutylisonitrile scintimammography: use of a new dedicated gamma camera. Eur J Nucl Med 1999; 26:1279-1288.

51. Hruska C B, O'Connor M K, Collins D A. Comparison of small field of view gamma camera systems for scintimammography. Nucl Med Commun 2005; 26: 441-445.

52. Brem R F, Schoonjans J M, Kieper D A, Majewski S, Goodman S, Civelek C. High resolution scintimammography: a pilot study. J Nucl Med 2002; 43:909-915.

53. Coover L R, Caravaglia G, Kuhn P. Scintimammography with dedicated breast camera detects and localizes occult carcinoma. J Nucl Med 2004; 45: 553-558.

54. Mueller B, O'Connor M K, Blevis I, Rhodes D J, Smith R, Collins D A, Phillips S W. Evaluation of a small CZT detector for scintimammography. J Nucl Med 2003; 44:602-609.

55. Rhodes D J, O'Connor M K, Phillips S W, Smith R L, Collins D A. Molecular Breast Imaging: A new technique using Tc-99m scintimammography for the detection of small breast lesions. Mayo Clinic Proc 2005; 80: 24-30.

1. A system for generating molecular breast images, the system comprising:
a cadmium-zinc-telluride (CZT) based gamma camera;
an upper CZT detector positioned in a first orientation;
a lower CZT detector positioned in an opposing orientation to the upper CZT detector;
user controls adapted for adjustment of the relative position of at least one of the upper CZT detector and the lower CZT detector, the adjustment serving as a subject breast compression mechanism to compress the subject breast to a total breast thickness; and
the upper CZT detector and the lower CZT detector positioned to reduce the maximum distance between a lesion in the subject breast and either the upper CZT detector and the lower CZT detector to about one half of the total breast thickness.

2. The system of claim 1 further including a processor for processing signals acquired by the upper CZT detector and the lower CZT detector.

3. The system of claim 2 wherein the signals acquired by the upper CZT detector and the lower CZT detector are processed by the processor to produce an image on a display.

4. The system of claim 1 further including a radiopharmaceutical for injection into the subject to be imaged.

5. The system of claim 4 wherein the radiopharmaceutical is selected from a group consisting of Tc-99m HIS₆-(Z_{HER2:4})₂ HER2 Receptor, Tc-99m Anti-CEA, In-111 satumomab pendetide (Oncoscint), Tc-99m depreotide, In-111 octretide, Tc-99m bombesin, I-123 tamoxifen, I-123 estradiol, Tc-99m sestamibi, Tc-99m tetrofosmin, Tc-99m annexin-V, In-111 vitamin B12, Tc-99m thio-glucose, Tc-99m glucarate, Tc-99m EC -deoxyglucose, Tc-99m exametazine, TI-201 Thallium Chloride, Tc-99m methylene diphosphonate, and Tc-99m (v) DMSA

6. A method for analyzing molecular breast images comprising the steps of:
a) injecting a radionuclide imaging into a subject to be imaged;
b) positioning a breast of the subject between a first and second opposing, planar gamma detectors;
c) compressing the breast to cause a breast thickness;
d) acquiring a number of photons from the breast at each of the gamma detectors to create at least one imaging data set, the first gamma detector acquiring photons from about a first half of the breast thickness, and the second gamma detector acquiring photons from about a second half of the breast thickness;
e) identifying a tumor in at least one of the imaging sets;
f) selecting a plurality of intensity profiles extending through the tumor from at least a portion of the imaging data sets; and
g) calculating a size metric of the tumor from the plurality of intensity profiles selected in step f).
